SEQUENCE LISTING (22 sequences)

SEQ ID NO: 1 - protein, 16 aa, Artificial Sequence
  Synthetic
GSLDESFYDWFERQLG

SEQ ID NO: 2 - protein, 10 aa, Artificial Sequence
  Synthetic
FYDWFERQLG

SEQ ID NO: 3 - protein, 5 aa, Artificial Sequence
  Synthetic
FYDWF

SEQ ID NO: 4 - protein, 5 aa, Artificial Sequence
  Synthetic
FYGWF

SEQ ID NO: 5 - protein, 10 aa, Artificial Sequence
  Synthetic
FYGWFERQLG

SEQ ID NO: 6 - protein, 16 aa, Artificial Sequence
  Synthetic
GSLDESFYGWFERQLG

SEQ ID NO: 7 - protein, 15 aa, Artificial Sequence
  Synthetic
GSLDESFYDWFERQL

SEQ ID NO: 8 - protein, 15 aa, Artificial Sequence
  Synthetic
GSLDESFYGWFERQL

SEQ ID NO: 9 - protein, 9 aa, Artificial Sequence
  Synthetic
FYDWFERQL

SEQ ID NO: 10 - protein, 9 aa, Artificial Sequence
  Synthetic
FYGWFERQL

SEQ ID NO: 11 - protein, 16 aa, Artificial Sequence
  Synthetic
XXXXXXFYXWFEXQLX

SEQ ID NO: 12 - protein, 21 aa, Artificial Sequence
  Synthetic
XLXXEWAXXQCEVXGRGCPSX

SEQ ID NO: 13 - protein, 6 aa, Artificial Sequence
  Synthetic
GGSGGS

SEQ ID NO: 14 - protein, 20 aa, Artificial Sequence
  Synthetic
WLDEEWAQVQCEVYGRGCPS

SEQ ID NO: 15 - protein, 20 aa, Artificial Sequence
  Synthetic
WLDQEWAWVQCEVYGRGCPS

SEQ ID NO: 16 - protein, 21 aa, Artificial Sequence
  Synthetic
SLEEEWAQIQCEVWGRGCPSY

SEQ ID NO: 17 - protein, 21 aa, Artificial Sequence
  Synthetic
XLXXEWAXXQCEVXGRGCPSX

SEQ ID NO: 18 - protein, 21 aa, Artificial Sequence
  Synthetic
XLXXEWAXXQCEVXGRGCPSX

SEQ ID NO: 19 - protein, 20 aa, Artificial Sequence
  Synthetic
SLEEEWAQIQCEVWGRGCPS

SEQ ID NO: 20 - protein, 37 aa, Artificial Sequence
  Synthetic
GSLDESFYDWFERQLXSLEEEWAQIQCEVWGRGCPSX

SEQ ID NO: 21 - protein, 43 aa, Artificial Sequence
  Synthetic
GSLDESFYDWFERQLGGGSGGSSLEEEWAQIQCEVWGRGCPSY

SEQ ID NO: 22 - protein, 43 aa, Artificial Sequence; identical to SEQ ID NO: 21
  Synthetic

(19) United States
(12) Patent Application Publication
(10) Pub. No.: US 2009/0197805 A1
(43) Pub. Date: Aug. 6, 2009

(54) Insulin receptor antagonists and related compositions, uses and methods

(75) Inventors: Lauge Schaffer, Lyngby (DK); Christian Lehn Brand, Allerod (DK); Ulla Ribel, Virum (DK); Jeppe Sturis, Vaerlose (DK)

Correspondence Address: NOVO NORDISK, INC.;INTELLECTUAL PROPERTY DEPARTMENT, 100 COLLEGE ROAD WEST, PRINCETON, NJ, 08540, US

(73) Assignee: Novo Nordisk A/S, Bagsvaerd (DK)

(21) Appl. No.: 12/089,222

(22) PCT Filed: Oct. 3, 2006
(86) PCT No.: PCT/EP06/66993

§ 371 Date: Aug. 8, 2008

(30) Foreign Application Priority Data
Oct. 5, 2005 (EP) 05109231.0

(51) Int. Cl. A61K 38/16; C07K 14/00; C12N 5/00; A61P 3/08

(52) U.S. Cl. 514/12; 530/324; 435/375

(57) ABSTRACT

Provided are new peptidic insulin receptor antagonists (PIRAs) and related compounds and compositions. Also provided are new uses of PIRAs and methods of obtaining PIRAs.

### FIELD OF THE INVENTION

This invention pertains to molecules that have insulin receptor antagonist activity, related pharmaceutical compositions, additional compositions related thereto, uses of such compositions and molecules, and various other related methods.

### BACKGROUND OF THE INVENTION

Insulin is one of the most studied peptide hormones because of its importance in maintaining glucose homeostasis. This 51-aa hormone is very well characterized with regard to its structure, both in crystal form and in solution.

The cellular response to insulin is mediated through the insulin receptor, which is a tetrameric protein consisting of two identical extracellular alpha-subunits which bind insulin and two identical transmembrane beta-subunits which have intracellular tyrosine kinase activity. Goldfine, Endocr. Rev., 8: 235 (1987). When insulin binds to the alpha-subunit, the beta-subunit tyrosine kinase is activated and insulin action ensues (resulting in a cascade of intracellular signaling events).

Hyperinsulinemia is a condition defined by abnormally high levels of insulin in the blood. Causes of hyperinsulinemia include insulinoma and insulin resistance, which may be caused by congenital hyperinsulinemia or other conditions, such as a lack of activity, obesity, or polycystic ovary syndrome. An insulinoma is a tumor of the pancreas that produces excessive amounts of insulin. High insulin levels cause hypoglycemia, or low blood glucose (sugar). Hyperinsulinemia is the most common cause of neonatal hypoglycemia following the first few hours of life. Treatment of such a condition may often be necessary to prevent onset of seizures and neurologic sequelae.

In general, hypoglycemia may be mild and lead to symptoms such as anxiety and hunger, but patients are also at risk for severe hypoglycemia, which can cause seizures, coma, and even death. Typical symptoms associated with hypoglycemia that patients complain about include tiredness, weakness, tremulous and hunger. Many patients have to eat frequently to prevent symptoms from the low blood sugar. Some patients may develop psychiatric symptoms because of the low blood sugar.

Currently, patients with insulinomas or other severe forms of hyperinsulinemia are treated by surgery such as partial pancreatectomy or by administration of drugs such as diazoxide or somatostatin which in some cases reduces insulin production. In some cases glucose must be infused continuously. There is no existing treatment which reduces the effect of circulating insulin.

Except for certain insulin receptor antibodies (see, e.g., Roth et al., J Biol Chem. 1983 Oct. 25; 258(20): 12094-7; Morgan et al., Proc Natl Acad Sci U S A. January 1986; 83(2): 328-32; Taylor et al., Biochem J. 1987 Feb. 15; 242(1): 123-9; Nagy et al., Endocrinology. January 1990; 126(1): 45-52; and Fujita et al., Acta Diabetol. December 2002; 39(4): 221-7), only a few insulin receptor (IR) antagonists have been previously described. See, e.g., U.S. Patent Publication Nos. 20030236190, 20030195147, and 20040023887 and Pillutla et al., J. Biol. Chem. 277 (25): 22590-22594, 2002 and Schaffer et al., Proc. Natl. Acad. Sci. U.S.A. 100 (8): 4435-4439, 2003.

### BRIEF SUMMARY OF THE INVENTION

There are several aspects to the invention described herein. In one exemplary aspect, the invention provides novel peptides, proteins, and protein derivatives that have insulin receptor antagonist activity. In another aspect, the invention provides pharmaceutical compositions comprising such novel peptidic molecules. In yet another aspect, the invention provides various additional useful compositions (e.g., nucleic acids comprising sequences encoding such peptides, vectors and cells comprising such nucleic acids, antibodies to such peptides and proteins, etc.). In still another aspect, the invention relates to new uses of such compositions and molecules (e.g., in screening/testing for disorders or conditions, in treatment of various disorders and conditions, in the production of antibodies for therapeutic and diagnostic applications, for the preparation of protein mimetics, etc.), and various other related methods. These and additional exemplary inventive aspects and features of the invention are described in further detail herein.

### BRIEF DESCRIPTION OF THE FIGURES

FIG. 1. Insulin stimulated glucose uptake in SGBS cells and inhibition of submaximally insulin stimulated glucose uptake by addition of S661

FIG. 2. Glucose profile in Zucker obese (ZO) male rats after intravenous (i.v.) administration of different doses of peptidic insulin receptor antagonist S661, followed by intraperitoneal (i.p.) administration of human insulin.

FIG. 3. Glucose profile in ZO rats after i.v. administration of Insulin Receptor Antagonist, S661, followed after different times by i.p. administration of human insulin.

### DETAILED DESCRIPTION OF THE INVENTION

Among other things, this invention relates to the discovery that certain new non-antibody peptides, proteins, and related protein derivatives (“peptidic molecules”), having certain physical features including comprising particular amino acid sequences, may be useful as peptidic insulin receptor (IR) antagonists (such peptidic molecules are often referred to as “peptidic insulin receptor antagonists” or “PIRAs” herein).

The invention further relates to novel pharmaceutically acceptable compositions comprising therapeutically effective amounts of one or more PIRAs of the invention (as the sole active pharmacological agent or in combination with one or more “secondary” agents or “second active agents”).

The invention also relates to new uses of such PIRAs, such as, for example, the production of medicaments using such PIRAs, and methods of treating conditions and disorders in which decreasing IR activity would be considered beneficial or useful.

These are, however, just some of the beneficial aspects of the invention. These and other aspects will be clear to skilled artisans given the description of the invention provided herein.

In general, PIRAs can be characterized as peptidic molecules comprising at least one “core motif” that comprises (a) a formula 1 or formula 1-like (“formula 1 type” or “F1T”) amino acid sequence (“F1TS”) and (b) a formula 6 or formula 6-like (“formula 6 type” or “F6T”) amino acid sequence (F6TS), wherein the F1TS and F6TS are located in proximity to one another in a peptide chain contained within (or that makes up) the PIRA, such that the PIRA binds to an IR and acts as an IR antagonist. F1T and F6T sequences are discussed in detail further herein. A PIRA may include many repeats of this F1TS/F6TS “core motif” or include more than one F1TS or F6TS, but typically a PIRA comprises a single F1TS and a single F6TS contained in a single “core motif”. In general, the discussion of F1T and F6T sequences provided herein is focused on F1T and F6T sequences contained in a “core motif.”

PIRAs can exhibit any suitable level of affinity for one or more IRs (preferably, a PIRA will exhibit affinity for the human IR). In one aspect, the invention provides PIRAs that have affinity for human IR that is comparable to the affinity exhibited by insulin for the human IR (e.g., the PIRA has an affinity for the human IR that is at least about 35%, at least about 40%, at least about 45%, at least about 50%, at least about 60%, at least about 70%, at least about 80%, at least about 90%, about 100% of the affinity, etc., of that of insulin).

PIRAs may or may not be characterized as being free of other functional domains. Thus, in one aspect, a PIRA may be characterized as a fusion protein comprising at least one second peptide domain that induces or promotes a physiological response (e.g., binding and modulation of a receptor other than an IR). In another aspect, the invention provides PIRAs that are free from such additional domains. In one aspect, the invention provides PIRAs that consist essentially of a single formula 1 sequence (or F1TS) and a single formula 6 sequence or F6TS (optionally separated by a suitable linker and/or associated with a suitable TDM).

“Antagonism” in the context of this invention refers to the ability of a compound (e.g., a PIRA) to detectably reduce the effects of insulin when in the presence of insulin and an IR. An antagonist may block the binding of insulin to the IR, reduce one or more insulin-mediated IR activities upon insulin binding (several examples of which have been well characterized), or both. In general, PIRAs may exhibit any detectable level of IR antagonism. In one aspect, the invention provides PIRAs that reduce the insulin-mediated IR activities by at least about 10%, at least about 20%, at least about 30%, at least about 40%, at least about 50%, at least about 60% or more.

In a particular aspect, the invention provides PIRAs that have the ability to increase blood glucose and/or reduce the effect of insulin in a mammalian host for a period of at least about 4 hours, at least about 5 hours, at least about 6 hours, at least about 7 hours, at least about 8 hours, or even at least about 9 hours.

Typically, the F6TS of the PIRA core motif is positioned C-terminal to the related F1TS (i.e., the core motif F6TS is positioned in the core motif in relation to the F1TS such that the N-terminal portion of the F6TS is located nearest to the C-terminal portion of the F1TS), such that PIRAs typically can be said to have a formula 1, formula 6-orientation (or F1/F6 orientation).

A PIRA generally can be of any suitable size, conformation, etc. PIRAs can be (and typically, and often advantageously, are) characterized as single chain peptides of about 50 amino acids or less in length, such as about 30-45 amino acids in length (e.g., 35-44 amino acids in length). However, larger IRA-containing proteins, multimeric proteins, multiple peptide-chain protein derivatives, or complex proteins, and the like, comprising PIRA portions or sequences of such a size and composition also can be generated using standard techniques and are considered another feature of the invention. PIRAs can be characterized in lacking the features of IR antibodies. Thus, for example, PIRAs typically do not comprise framework region and CDR sequences found in IR antibodies. Moreover, PIRAs typically lack the structure of known anti-IR antibodies (e.g., in lacking the classic tetrameric structure of an IgG molecule).

A formula 1 sequence is defined as an amino acid sequence comprising the sequence GSLDESFYDWFERQLG (Gly Ser Leu Asp Glu Ser Phe Tyr Asp Trp Phe Glu Arg Gln Leu Gly) (SEQ ID NO:1) (“Formula 1”). A formula 1-like sequence can be defined as a sequence that is very similar to SEQ ID NO:1 (i.e., a sequence having at least about 60%, at least about 65%, at least about 70%, at least about 75%, at least about 80%, at least about 85%, at least about 90%, or at least about 95% identity, but less than 100% identity, to SEQ ID NO:1 (such similar sequences may be referred to as Formula 1-like sequences)), that which, can be combined in a core motif with a F6TS to produce a PIRA. In one aspect, the invention provides PIRAs wherein about 1-6 of the N-terminal residues of Formula 1 are deleted and/or substituted (e.g., residues 1-5 are deleted, residues 2-6 are deleted, residues 1-4 are deleted and residues 5 and 6 substituted with other suitable residues, etc.). Thus, for example, in one aspect, the invention provides PIRAs comprising a F1TS that consists essentially of the sequence FYDWFERQLG (SEQ ID NO:2). In another aspect, the invention provides PIRAs that simply are characterized by comprising a sequence containing the motif FYDWF (SEQ ID NO:3). In another aspect, the invention provides F1TSs that may also or alternatively be characterized by having a sequence marked by the substitution of the Formula 1 Asp_{g} residue (or a residue corresponding thereto) with a Gly residue (or similar equivalent residue), such that PIRA comprises the motif FYGWF (SEQ ID NO:4), for example in the context of a F1TS consisting essentially of FYGWFERQLG (SEQ ID NO:5) or GSLDESFYGWFERQLG (SEQ ID NO:6). In yet another aspect, the invention provides PIRAs comprising a F1TS that also (in addition to one or more of the foregoing characteristics) or alternatively is characterized by lack of, or substitution of, Formula 1 residue Gly₁₆ (or residue corresponding thereto). GSLDESFYDWFERQL (SEQ ID NO:7), GSLDESFYGWFERQL (SEQ ID NO:8), FYDWFERQL (SEQ ID NO:9), and FYGWFERQL (SEQ ID NO:10) are examples of such F1TSs. In still another aspect, the invention provides PIRAs comprising a F1TS that also (i.e., in addition to one or more of the foregoing characteristics) or alternatively is characterized by the deletion of or substitution of Arg₁₃ of Formula 1 (or corresponding residue). As already indicated, these various specific modifications can be combined, such that the invention provides, for example, PIRAs characterized by comprising a F1TS according to the formula Xaa₂₂Xaa₂₃Xaa₂₄Xaa₂₅Xaa₂₆Xaa₂₇Phe Tyr Xaa₃₀ Trp Phe Glu Xaa₃₄ Gln Leu Xaa₃₇ (SEQ ID NO:11), wherein Xaa₂₂ is an optionally present Gly residue; Xaa₂₃ is an optionally present Ser residue; Xaa₂₄ is an optionally present Leu residue; Xaa₂₅ is an optionally present Asp residue; Xaa₂₆ is an optionally present Glu residue; Xaa₂₇ is an optionally present Ser residue; Xaa₃₀ is an Asp or Gly residue; Xaa₃₄ is any suitable amino acid residue (in one aspect Xaa₃₄ is selected from Gln, His, Lys, Glu, Asn, Asp, and Ser; in another aspect Xaa₃₄ is selected from Ala, Cys, Phe, His, lie, Leu, Met, Thr, Val, Trp, and Tyr; and in yet another aspect Xaa₃₄ is selected from Glu, Gln, Thr, Lys, Ser, Gly, Pro, Asp, and Glu), but typically is Arg (though in one aspect is characterized as not being Arg); and Xaa₃₇ is an optionally present Gly (Formula 1A).

Identity in the context of amino acid sequences of the invention can be determined by a Needleman-Wunsch alignment analysis (see Needleman and Wunsch, J. Mol. Biol. (1970) 48: 443-453), such as by analysis with ALIGN 2.0 using the BLOSUM50 scoring matrix with an initial gap penalty of −12 and an extension penalty of −2 (see Myers and Miller, CABIOS (1989) 4: 11-17 for discussion of the global alignment techniques incorporated in the ALIGN program). A copy of the ALIGN 2.0 program is available through the San Diego Supercomputer (SDSC) Biology Workbench. Because Needleman-Wunsch alignment provides an overall or global identity measurement between two sequences, it should be recognized that target sequences which may be portions or subsequences of larger peptide sequences may be used in a manner analogous to complete sequences or, alternatively, local alignment values can be used to assess relationships between subsequences, as determined by, e.g., a Smith-Waterman alignment (J. Mol. Biol. (1981) 147: 195-197), which can be obtained through available programs (other local alignment methods that may be suitable for analyzing identity include programs that apply heuristic local alignment algorithms such as FastA and BLAST programs). Further related methods for assessing identity are described in, e.g., International Patent Application WO 03/048185.

In addition to possessing a F1TS, PIRAs are further characterized by comprising at least one (and typically only one) amino acid sequence according to the formula Xaa Leu Xaa Xaa Glu Trp Ala Xaa Xaa Gln Cys Glu Val Xaa Gly Arg Gly Cys Pro Ser Xaa (SEQ ID NO:12), wherein Xaa represents any suitable amino acid residue (Formula 6).

In a particular aspect of the invention, the F1T sequence and the F6T sequence of a PIRA directly contact one another.

In a particular and often advantageous aspect of the invention, the invention provides PIRAs that comprise a F1T sequence and a F6T sequence (which F6T sequence is positioned C-terminal to the F1T sequence), wherein the F1T sequence and F6T sequence are separated by a linker. In one aspect, the linker is a non-amino acid moiety. Examples of such moieties include aliphatic or aromatic carbon chains and linkers based on one or more polyethylene glycol (PEG) units; etc. (examples of such chemical linkers and other suitable linker moieties can be found in, e.g., U.S. patent applications 20030195147, 20030236190, 20040023887, and International Patent Applications PCT/EP2005/054095 and WO 2005082404). In a particular often advantageous aspect, the invention provides PIRAs that comprise a F1T sequence and a F6T sequence (in formula 1, formula 6-orientation) wherein the F1T sequence and F6T sequence are separated by one or more amino acid residues. In a particular aspect, the F1T sequence (F1TS) and F6T sequence (F6TS) are separated by 1 -10 amino acid residues, such as 1-8 amino acid residues, such as 1-7, 2-7, 2-6,1-6, 2-5,1-5, 3-6, 3-7, or 3-5 amino acid residues or by any number of residues comprised within such ranges (e.g., 1, 2, 3, 4, 5, 6, or 7 residues). Unless otherwise specified, any suitable amino acid sequence linker can be used to join a F1TS and F6TS. The qualities of suitable linker sequences are well known in the art and, accordingly, do not need to be discussed in detail here. In general, an amino acid sequence linker is typically primarily composed (at least 50%, at least 70%, at least 85%, at least 90% composed, etc.) of “flexible” amino acid residues. Naturally occurring amino acid residues that are considered flexible and often used alone or in combination as linker sequences include serine residues, glycine residues, and the like (e.g., in one aspect the invention provides PIRAs that comprise a linker comprising a sequence according to the formula Xaa_{L1}Xaa_{L2}Xaa_{L3}Xaa_{L4}Xaa_{L5}Xaa_{L6}, wherein each of Xaa_{L1}Xaa_{L2}Xaa_{L3}Xaa_{L4}Xaa_{L5}Xaa_{L6} are independently Gly or Ser (an example of such a linker is embodied in a linker comprising the sequence Gly Gly Ser Gly Gly Ser (SEQ ID NO:13)). However, Gln, Thr, Lys, Pro, Asp, Phe, and Arg residues also may be considered sufficiently “flexible,” though typically inclusion of a number of such residues, if any, is less preferred. Synthetic and modified amino acid residues having similar “flexibility” also may be used. Some percentage of the residues in a linker also may be drawn from “non-flexible” residues, for example where the linker is to be imparted with other biological functions. Various non-common amino acid residues that may be suitably incorporated into PIRAs in this and other contexts are known in the art and described in references incorporated herein, and selection of residues with desirable properties is within the skill of the ordinary artisan using only routine experimentation. For example, where an alpha-helical linker is desired, unnatural amino acids such as e.g. Aib (aminoisobutyric acid) can be used, and if a beta-turn is desired the linker could contain either a Gly-Pro sequence or an unnatural sequence. In general, an amino acid sequence linker is limited to amino acids, and does not comprise any non-amino acid linker moieties. In alternative aspects, a linker composed of one or more amino acid residues and non-amino acid linking moieties can be used.

In one aspect, the invention provides PIRAs that comprise a F1T sequence and a F6T sequence (in formula 1, formula 6-orientation) wherein the PIRA is characterized by comprising a terminal derivatizing moiety (“TDM”). In general, a terminal derivatizing moiety can be any derivatizing moiety that promotes stability of the PIRA in vivo (i.e., in a mammalian host) and/or that provides the PIRA with other desired properties such as improved solubility or improved pharmaceutical stability. Measurement of such properties can be accomplished by routinely used methodologies.

Typical TDMs include acyl (e.g., acetyl) and amide groups (in other words, typical TDM-associated PIRAs comprise at least one (and typically only one or two) acyl-derivatized or amide-derivatized amino acid residue). A TDM-comprising PIRA also may optionally be characterized by comprising a linker separating the F1TS and F6TS, such as an amino acid sequence linker. Typically, any type of PIRA comprising a TDM will comprise a TDM positioned at (either directly or via a linker (either an amino acid sequence linker or non-amino acid sequence linker), but typically directly) the C-terminus of the F6TS (and usually a PIRA will comprise only one TDM so positioned), usually wherein the terminus of the F6TS constitutes the terminus of the PIRA.

In a particular aspect, the invention provides PIRAs comprising a F1TS and a F6TS, in formula 1, formula 6-orientation, wherein the F1TS and F6TS are joined or separated by an amino acid sequence linker, wherein the F6TS is characterized as not being selected from WLDEEWAQVQCEVYGRGCPS (SEQ ID NO:14). In another aspect, the invention provides PIRAs having such features, wherein the F6TS is characterized as not being selected from WLDEEWAQVQCEVYGRGCPS or WLDQEWAWVQCEVYGRGCPS (SEQ ID NO:15). In still another aspect, the invention provides PIRAs having a FT6 sequence, which is characterized as having about 95% or less, about 90% or less, about 85% or less, about 80% or less, or even about 75% or less identity to SEQ ID NO:14, SEQ ID NO:15, or both SEQ ID NO:14 and SEQ ID NO:15. In a more particular aspect, the invention provides PIRAs comprising a F6TS that has 70-95%, 75-95%, 80-95%, 85-95%, 90-95%, 70-90%, 75-90%, 75-85%, 75-80%, 80-90%, 80-85%, about 80%, or about 85% identity to SEQ ID NO:14 or SEQ ID NO:15. Such PIRAs may optionally be further characterized by comprising one or more TDMs.

In another aspect, the invention provides PIRAs that comprise a F6TS that is characterized as having about 70-95% identity, such as about 75-95%, 80-95%, 85-95%, 90-95%, 90%, 95%, 85%, 80%, 75%, 75-90%, or 80-90% identity to SLEEEWAQIQCEVWGRGCPSY (SEQ ID NO:16). Such PIRAs may optionally be associated with a linker between the F1TS and F6TS, which typically is an amino acid sequence linker. Such PIRAs may also or alternatively be associated with one or more TDMs. For example, such a PIRA may comprise a single TDM located at the C-terminal Tyr residue of the F6TS (which C-terminal residue usually serves as the C-terminus of the PIRA) or a residue that substitutes for the Tyr residue or another proximate terminal residue (e.g., a residue that would be considered to “correspond” to Ser₂₀ in the case of such a F6TS wherein the F6TS lacks Tyr₂₁).

In one aspect, the invention provides PIRAs that comprise a F6TS wherein one or both of the Cys residues contained in Formula 6 (or corresponding Cys residues in a F6TS) are deleted or substituted. Such a F6TS may also exhibit any feature or combination of features described herein with respect to F6TSs (e.g., otherwise comprising a sequence according to Formula 6a (discussed below)—i.e., the invention provides PIRAs comprising a sequence according to Formula 6a, but wherein one or both of the Cys residues thereof are, independently (if both Cys residues are affected), deleted or substituted with another suitable residue or moiety).

In another aspect, the invention provides PIRAs characterized by retention of two Cys residues positioned similarly to the Cys residues of Formula 6. Such a F6TS may exhibit any feature or combination of features described herein with respect to F6TSs.

In another exemplary and particular aspect, the invention provides PIRAs that comprise a F6TS according to the formula Xaa₁Leu Xaa₃Xaa₄ Glu Trp Ala Xaa₈ Xaa₉ Gln Cys Glu Val Xaa₁₄ Gly Arg Gly Cys Pro Ser Xaa₂₁ (SEQ ID NO:17), wherein Xaa₁ is any suitable amino acid residue; Xaa₃ is an Asp, Glu, or another suitable acidic residue; Xaa₄ is any suitable amino acid residue (typically a residue selected from C, D, E, H, K, N, Q, R, S, and T; more typically a residue selected from N, E, Q, H, R, and K; and even more typically a residue selected from N, E, Q, and K); Xaa₈ is any suitable amino acid residue; Xaa₉ is any suitable amino acid residue (but typically is selected from I, L, V, M, F, G, and A; more typically from I, L, V, and M; and even more typically from I, L, or V); Xaa₁₄ is a cycloalkenyl-associated amino acid residue (e.g., W, F, Y or H; typically W, F, or Y); and Xaa₂₁ is either absent or is an uncharged hydrophilic or polar amino acid residue (e.g., S, T, N, Q, Y, C, or G) (Formula 6a). PIRAs comprising Formula 6a sequences may be characterized by comprising a TDM, as discussed above, and/or by including a linker (e.g., an amino acid sequence linker) between the F1TS and Formula 6a sequence (as described above). Formula 6a PIRAs also or alternatively can be characterized by the limitation that the Formula 6a sequence is not WLDEEWAQVQCEVYGRGCPS (i.e., SEQ ID NO:14), WLDQEWAWVQCEVYGRGCPS (i.e., SEQ ID NO:15), or SLEEEWAQIQCEVWGRGCPSY (i.e., SEQ ID NO:16), and in a more particular aspect such PIRAs may be characterized by the fact that the Formula 6a sequence contained therein is about 70-95%, such as about 75-90%, such as about 75-85%, such as about 75-80%, such as about 80-90%, such as about 75%, 80%, 85%, or 90% identical to one or more of SEQ ID NOs:14-16.

In still another exemplary and particular aspect, the invention provides PIRAs that comprise a F6TS according to the formula Xaa₁Leu Xaa₃Xaa₄ Glu Trp Ala Xaa₈Xaa₉Gln Cys Glu Val Xaa₁₄ Gly Arg Gly Cys Pro Ser Xaa₂₁ (SEQ ID NO:18), wherein Xaa₁ is Trp or Ser, Xaa₃ is Glu or Asp; Xaa₄ is Glu or Gln; Xaa₈ is Gln or Trp; Xaa₉ is IIe or Val; Xaa₁₄ is Trp or Tyr; and Xaa₂₁ is absent or Tyr (Formula 6b).

PIRAs comprising a Formula 6b sequence may be characterized by comprising a TDM, as discussed above, and/or by including a linker (e.g., an amino acid sequence linker) between the F1TS and Formula 6b sequence (as described above). Formula 6b PIRAs also or alternatively can be characterized by the limitation that the Formula 6b sequence is not WLDEEWAQVQCEVYGRGCPS (i.e., SEQ ID NO:14), WLDQEWAWVQCEVYGRGCPS (i.e., SEQ ID NO:15), or SLEEEWAQIQCEVWGRGCPSY (i.e., SEQ ID NO:16), and in a more particular aspect such PIRAs may be characterized by the fact that the Formula 6b sequence contained therein is about 70-95%, such as about 75-90%, such as about 75-85%, such as about 75-80%, such as about 80-90%, such as about 75%, 80%, 85%, or 90% identical to one or more of SEQ ID NOs:14-16.

In another aspect, the invention provides PIRAs comprising a F6TS selected from WLDEEWAQVQCEVYGRGCPS (i.e., SEQ ID NO:14), WLDQEWAWVQCEVYGRGCPS (i.e., SEQ ID NO:15), and SLEEEWAQIQCEVWGRGCPS (i.e., SEQ ID NO:16). Like other PIRAs described herein, such PIRAs may comprise a linker between the F1TS and F6TS (e.g., an amino acid sequence linker) and/or a TDM (typically positioned at the C-terminus of the F6TS or at a residue located near the end of the F6TS).

In another aspect, the invention provides PIRAs characterized by comprising one or more of the previously described features, wherein the formula 6-type sequence of the PIRA does not comprise the sequence SLEEEWAQIQCEVWGRGCPS (SEQ ID NO:19).

In a more particular exemplary aspect, the invention provides PIRAs comprising an amino acid sequence (core motif sequence) according to the formula GSLDESFYDWFERQL-Z₁-SLEEEWAQIQCEVWGRGCPS-Z₂ (SEQ ID NO:20), wherein Z₁ represents an amino acid linker and Z₂ represents an optionally present terminal derivatizing moiety, an amino acid or amino acid sequence (e.g., a stabilizing sequence, such as an albumin or albumin-derived sequence), a derivatized amino acid residue, or an amino acid sequence comprising a C-terminal derivatized amino acid residue.

A particular exemplary PIRA provided by the invention is GSLDESFYDWFERQLGGGSGGSSLEEEWAQIQCEVWGRGCPSY-amide (“S661”).

In yet another aspect, the invention provides PIRAs that comprise a core motif that has about 65-95% identity, such as about 70-95%, 75-95%, 80-95%, 85-95%, 90-95%, 65-90%, 70-90%, 75-90%, 85-90%, 65-85%, 70-85%, 75-85%, 65%, 70%, 75%, 80%, 85%, or 90% identity to GSLDESFYDWFERQLGGGSGGSSLEEEWAQIQCEVWGRGCPSY (SEQ ID NO:19). Such “S661-like PIRAs” may be characterized by comprising a TDM, which typically is associated with the C-terminal Tyr residue (or residue that would be considered to “correspond” thereto—as determined by, e.g., sequence alignment analysis).

PIRAs can be prepared by any suitable method. For example, PIRAs, particularly non-derivative PIRAs, can be produced as fusion proteins in any suitable expression system. Methods and principles relevant to the production of recombinant fusion proteins are very well known in the art and need not be discussed in detail here. Standard peptide synthesis (e.g., solid phase peptide synthesis) can be used to generate PIRAs as well. Such recombinantly produced or synthesized peptides can further be subjected to derivation, conjugation, multimerization, etc. to form more complicated molecules within the scope of this invention. PIRAs likewise can be purified by any suitable technique. For example, “PIRA fusion proteins” comprising, e.g., particular purification “tags” (purification facilitating sequences or moieties) can be generated by known methods and used as a means for obtaining a purified PIRA molecule. For direct purification of non-fusion protein PIRAs, methods such as differential electrophoresis, chromatography, centrifugation also can be used as can affinity (e.g., antibody-based) methods directed to the characteristics of the non-fusion protein PIRA.

PIRAs also include protein “derivatives” (or “PIRA derivatives”), which include, but are not limited to, the TDM-associated PIRAs described above. The term “derivative” generally refers to a protein or peptide in which one or more of the amino acid residues contained therein have been chemically modified (e.g., by alkylation, acylation, ester formation, amide formation, or other similar type of modification) or covalently associated with one or more other heterologous substituents (e.g., a lipophilic substituent, a PEG moiety, a peptide side chain linked by a suitable organic moiety linker, etc.).

In general, PIRAs described herein can be modified by inclusion of any suitable number of such modified amino acids and/or associations with such conjugated substituents. Suitability in this context generally is determined by the ability of the derivative to act as an IR antagonist with respect to a corresponding “naked” (i.e., non-derivatized) counterpart PIRA. The inclusion of one or more modified amino acids may be advantageous in, for example, (a) increasing polypeptide serum half-life, (b) reducing polypeptide antigenicity, or (c) increasing polypeptide storage stability. Amino acid (s) are modified, for example, co-translationally or post-translationally during recombinant production (e.g., N-linked glycosylation at N—X—S/T motifs during expression in mammalian cells) or modified by synthetic means. Non-limiting examples of a modified amino acid include a glycosylated amino acid, a sulfated amino acid, a prenlyated (e.g., famesylated, geranylgeranylated) amino acid, an acetylated amino acid, an acylated amino acid, a PEGylated amino acid, a biotinylated amino acid, a carboxylated amino acid, a phosphorylated amino acid, and the like. References adequate to guide one of skill in the modification of amino acids are replete throughout the literature. Exemplary protocols are found in, e.g., Walker (1998) PROTEIN PROTOCOLS ON CD-ROM Humana Press, Towata, N.J. Typically, the modified amino acid is selected from a glycosylated amino acid, a PEGylated amino acid, a famesylated amino acid, an acylated amino acid, a biotinylated amino acid, an amino acid conjugated to a lipid moiety, and an amino acid conjugated to an organic derivatizing agent.

PIRAs can be chemically modified by covalent conjugation to a polymer to increase their circulating half-life, for example. Exemplary polymers and methods to attach such polymers to peptides are illustrated in, e.g., U.S. Pat. Nos. 4,766,106; 4,179,337; 4,495,285; and 4,609,546. Additional illustrative polymers include polyoxyethylated polyols and polyethylene glycol (PEG) moieties (e.g., a PIRA can be conjugated to a PEG with a molecular weight of between about 1,000 and about 40,000, such as between about 2000 and about 20,000, e.g., about 3,000-12,000, and even more particularly about 5,000).

Thus, the peptides of the invention may be subjected to one or more modifications known in the art, which may be useful for manipulating storage stability, pharmacokinetics, and/or any aspect of the bioactivity of the peptide, such as, e.g., potency, selectivity, and drug interaction. Chemical modification to which the peptides may be subjected includes, without limitation, the conjugation to a peptide of one or more of polyethylene glycol (PEG), monomethoxy-polyethylene glycol, dextran, poly-(N-vinyl pyrrolidone) polyethylene glycol, propylene glycol homopolymers, a polypropylene oxide/ethylene oxide co-polymer, polypropylene glycol, polyoxyethylated polyols (e.g., glycerol) and polyvinyl alcohol, colominic acids or other carbohydrate based polymers, polymers of amino acids, and biotin derivatives. PEG conjugation of proteins at Cys residues is disclosed, e.g., in Goodson, R. J. & Katre, N. V. (1990) Bio/Technology 8, 343 and Kogan, T. P. (1992) Synthetic Comm. 22, 2417.

Other useful modifications include, without limitation, acylation, which may be performed using standard and know methods, e.g., using methods and compositions such as described in, e.g., U.S. Pat. Ser. No. 6,251, 856, and WO 00/55119. In one aspect, the invention provides a method of preparing a long-acting (highly stable) PIRA derivative and to such derivatized PIRAs. In one facet, a long acting PIRA is obtained by preparing a fusion protein comprising a “PIRA portion” and one or more “fusion partner portions” that comprise domains, sequences, etc., associated with increased in vivo stability. Examples of such fusion partner sequences include sequences derived from albumin, transferrin, and antibody sequences. Examples of albumin fusion proteins, methods of producing such fusion proteins, and suitable albumin fusion partner sequences are described in, e.g., U.S. Pat. Nos. 5,876,969 and 5,766,883. In another facet, a long acting PIRA is obtained by derivatizing a PIRA by chemical modification, such as acylation (through an appropriate linker) which could, e.g., lead to strong albumin binding with the possibility of acting as a PIRA while bound to albumin. The latter could also be achieved by modifying the peptide sequence or extending it with a sequence which is able to bind to a larger protein (such as, e.g., osteogenic growth peptide (OGP), which binds to alpha2-macroglobulin).

PIRAs can be provided in a homogenous composition or in combination with other active and/or inert ingredients.

PIRAs typically are used in and provided in an at least substantially pure form. A “substantially pure” molecule is a molecule that is the predominant species in the composition wherein it is found with respect to the class of molecules to which it belongs (e.g., a substantially pure protein is the predominant protein species in the composition wherein it is found). A substantially pure species makes up at least about 50% of the type of molecule in the composition and typically will make up at least about 70%, at least about 80%, at least about 85%, at least about 90%, at least about 95%, or greater percentage of the molecular species in the composition by weight. Commonly, a composition comprising a PIRA will exhibit at least about 98%, 98%, or 99% homogeneity for the PIRA in the context of all present peptide species in the composition or at least with respect to substantially active peptide species in the context of proposed use. For example, a peptide stabilizer/buffer such as an albumin may be intentionally included in a final pharmaceutical formulation, without impeding the activity of the PIRAs, and, accordingly, may be excluded from such purity calculations. The presence of impurities that do not interfere with the fundamental activity also may be acceptable in the context of a substantially pure composition. Purity can be measured by methods appropriate for the given compound (e.g., chromatographic methods; agarose and/or polyacrylamide gel electrophoresis; HPLC analysis; etc.).

In one aspect, the invention relates to compositions comprising an “isolated” PIRA. An “isolated molecule” refers to a molecule that is not associated with significant levels (e.g., more than about 1%, more than about 2%, more than about 3%, or more than about 5%) of any extraneous and undesirable biological molecules, such as non-PIRA biological molecules contained within a cell, cell culture, chemical media, or animal in which the PIRA was produced. An isolated molecule also refers to any molecule that has passed through such a stage of purity due to human intervention (whether automatic, manual, or both) for a significant amount of time (e.g., at least about 10 minutes, at least about 20 minutes, at least about one hour, or longer). In many of the various compositions provided by the invention, such as in a composition comprising one or more pharmaceutically acceptable carriers, a PIRA can be present in relatively small amounts in terms of numbers of total molecular species in the composition (e.g., in the case of a composition comprising a large amount of a pharmaceutically acceptable carrier, stabilizer, and/or preservative). In some cases additional peptides, such as bovine serum albumin (BSA), can be included in such a composition with a previously purified PIRA. However, provided that such additional constituents of the composition are acceptable for the intended application of the PIRA, such a composition can still be described as comprising an isolated PIRA. In other words, the term “isolated” is not meant to exclude artificial or synthetic mixtures with other compounds or materials, such as may form part of a pharmaceutically acceptable preparation.

In one aspect, the invention provides compositions comprising one or more PIRAs that are substantially free of other IR-binding molecules.

In another aspect, the invention provides a composition comprising a number of PIRAs with different characteristics (e.g., the invention provides in one aspect a “cocktail” of PIRAs having different characteristics).

PIRA compositions for pharmaceutical use typically contain at least a physiologically effective amount and commonly desirably contain a therapeutically effective amount of a PIRA, combination of PIRAs, or one or more PIRA(s) and additional active/therapeutic agents.

Terms such as a “therapeutically effective amount” refer to an amount of a biologically active compound or composition that, when delivered in appropriate dosages and for appropriate periods of time to a host that typically is responsive for the compound or composition, is sufficient to achieve a desired therapeutic result in a host and/or typically able to achieve such a therapeutic result in substantially similar hosts (e.g., patients having similar characteristics as a patient to be treated). A therapeutically effective amount of a PIRA may vary according to factors such as the disease state, age, sex, and weight of the individual, and the ability of the PIRA to elicit a desired response in the individual. A therapeutically effective amount is also one in which any toxic or detrimental effects of the agent are outweighed by the therapeutically beneficial effects. Exemplary therapeutic effects include, e.g., (a) a reduction in the severity of a disease, disorder, or related condition in a particular subject or a population of substantial similar subject; (b) a reduction in one or more symptoms or physiological conditions associated with a disease, disorder, or condition; and/or (c) a prophylactic effect. A reduction of the severity of a disease can include, for example, (a) a measurable reduction in the spread of a disorder; (b) an increase in the chance of a positive outcome in a subject (e.g., an increase of at least about 5%, 10%, 15%, 20%, 25%, or more); (c) an increased chance of survival or lifespan; and/or (d) a measurable reduction in one or more biomarkers associated with the presence of the disease state (e.g., a reduction in the amount and/or severity of symptoms; etc.). A therapeutically effective amount can be measured in the context of an individual subject or, more commonly, in the context of a population of substantial similar subjects (e.g., a number of human patients with a similar disorder enrolled in a clinical trial involving a PIRA composition or a number of non-human mammals having a similar set of characteristics being used to test a PIRA in the context of preclinical experiments).

A suitable dose of a PIRA for human therapeutic applications/regimens is expected to be in the range of about 1-200 nmol/kg, e.g., about 1-100 nmol/kg.

PIRAs also can be delivered to a host in a prophylactically effective amount as part of a disease/disorder prevention program or for otherwise increasing general health. A “prophylactically effective amount” refers to an amount of an active compound or composition that is effective, at dosages and for periods of time necessary, in a host typically responsive to such compound or composition, to achieve a desired prophylactic result in a host or typically able to achieve such results in substantially similar hosts. Exemplary prophylactic effects include a reduction in the likelihood of developing a disorder, a reduction in the intensity or spread of a disorder, an increase in the likelihood of survival during an imminent disorder, a delay in the onset of a disease condition, a decrease in the spread of an imminent condition as compared to in similar patients not receiving the prophylactic regimen, etc. Typically, because a prophylactic dose is used in subjects prior to or at an earlier stage of disease, the prophylactically effective amount will be less than the therapeutically effective amount for a particular PIRA. A prophylactic effect also can include, e.g., a prevention of the onset, a delay in the time to onset, a reduction in the consequent severity of the disease as compared to a substantially similar subject not receiving PIRA composition, etc.

In another aspect, PIRAs can be delivered to a host or cells in a “physiologically effective” amount. A physiologically effective amount is an amount of an active agent that upon administration to a host that is normally responsive to such an agent results in the induction, promotion, and/or enhancement of at least one physiological effect associated IR antagonism.

A PIRA can be combined with one or more carriers (diluents, excipients, and the like) appropriate for one or more intended routes of administration to provide compositions that are pharmaceutically acceptable in the context of preparing a pharmaceutically acceptable composition comprising one or more PIRAs.

PIRAs may be, for example, admixed with lactose, sucrose, powders (e.g., starch powder), cellulose esters of alkanoic acids, stearic acid, talc, magnesium stearate, magnesium oxide, sodium and calcium salts of phosphoric and sulphuric acids, acacia, gelatin, sodium alginate, polyvinylpyrrolidine, and/or polyvinyl alcohol, and optionally further tabletted or encapsulated for conventional administration. Alternatively, a PIRA may be dissolved in saline, water, polyethylene glycol, propylene glycol, carboxymethyl cellulose colloidal solutions, ethanol, corn oil, peanut oil, cottonseed oil, sesame oil, tragacanth gum, and/or various buffers. Other carriers, adjuvants, and modes of administration are well known in the pharmaceutical arts. A carrier or diluent may include time delay material, such as glyceryl monostearate or glyceryl distearate alone or with a wax, or other functionally similar materials.

Pharmaceutically acceptable carriers generally also include any and all suitable solvents, dispersion media, coatings, antibacterial and antifungal agents, isotonic and absorption delaying agents, and the like that are physiologically compatible with a PIRA. Examples of pharmaceutically acceptable carriers include water, saline, phosphate buffered saline (PBS), dextrose, glycerol, ethanol, and the like, as well as combinations of any thereof. In many cases, it can be desirable to include isotonic agents, for example, sugars, polyalcohols such as mannitol, sorbitol, or sodium chloride in such a composition. Pharmaceutically acceptable substances such as wetting agents or minor amounts of auxiliary substances such as wetting agents or emulsifying agents, preservatives or buffers, which desirably can enhance the shelf life or effectiveness of the PIRA, related composition, or combination. Suitability for carriers and other components of pharmaceutical compositions can be determined based on the lack of significant negative impact on the desired biological properties of the PIRA, related composition, or combination (e.g., less than an about 20%, 15%, 10%, 5%, or 1% reduction in IR antagonism).

PIRA compositions, related compositions, and combinations according to the invention may be presented, prepared, and/or administered in a variety of suitable forms. Such forms include, for example, liquid, semi-solid and solid dosage forms, such as liquid solutions (e.g., injectable and infusible solutions), dispersions or suspensions, emulsions, microemulsions, tablets, pills, powders, liposomes, dendrimers and other nanoparticles (see, e.g., Baek et al., Methods Enzymol. 2003; 362: 240-9; Nigavekar et al., Pharm Res. March 2004; 21 (3):476-83), microparticles, and suppositories. The optimal form for any PIRA-associated composition depends on the intended mode of administration, the nature of the composition or combination, and therapeutic application or other intended use. Formulations also can include, for example, powders, pastes, ointments, jellies, waxes, oils, lipids, lipid (cationic or anionic) containing vesicles, DNA conjugates, anhydrous absorption pastes, oil-in-water and water-in-oil emulsions, emulsions, carbowax (polyethylene glycols of various molecular weights), semi-solid gels, and semi-solid mixtures containing carbowax. Any of the foregoing mixtures may be appropriate in treatments and therapies in accordance with the present invention, provided that the antagonism of the IR by the PIRA is not significantly inhibited by the formulation and the formulation is physiologically compatible and tolerable with the route of administration. See also, e.g., Powell et al. “Compendium of excipients for parenteral formulations” PDA J Pharm Sci Technol. 52: 238-311 (1998) and the citations therein for additional information related to excipients and carriers well known to pharmaceutical chemists. In a particular aspect, PIRAs may be formulated and administered in liposomes. In another aspect, PIRAs are administered in liposomes with one or more secondary agents.

PIRA compositions also include compositions comprising any suitable combination of a PIRA peptide and an associated salt. Any suitable salt, such as an alkaline earth metal salt in any suitable form (e.g., a buffer salt), can be used in the stabilization of PIRAs (preferably the amount of salt is such that oxidation and/or precipitation of the PIRA is avoided). Suitable salts typically include sodium chloride, sodium succinate, sodium sulfate, potassium chloride, magnesium chloride, magnesium sulfate, and calcium chloride. Compositions comprising a base and one or more PIRAs also are provided.

A typical mode for delivery of PIRA compositions is by parenteral administration (e.g., intravenous, subcutaneous, intraperitoneal, and/or intramuscular administration). In one aspect, a PIRA is administered to a human patient by intravenous infusion or injection. In another aspect, a PIRA is administered by intramuscular or subcutaneous injection. Intratumoral administration also may be useful in certain therapeutic regimens (e.g., in the case of insulinoma treatment).

PIRAs may be formulated in, for example, solid formulations (including, e.g., granules, powders, projectile particles, or suppositories), semisolid forms (gels, creams, etc.), or in liquid forms (e.g., solutions, suspension, or emulsions). PIRAs may be applied in a variety of solutions. Suitable solutions for use in accordance with the invention typically are sterile, dissolve sufficient amounts of the PIRA and other components of the composition, stable under conditions for manufacture and storage, and not harmful to the subject for the proposed application. A PIRA may be subjected to conventional pharmaceutical operations such as sterilization and/or may contain conventional adjuvants, such as preservatives, stabilizers, wetting agents, emulsifiers, buffers etc. A composition also can be formulated as a solution, microemulsion, dispersion, powder, macroemulsion, liposome, or other ordered structure suitable to high drug concentration. Desirable fluidity properties of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin. These and other components of a pharmaceutically acceptable composition of the invention can impart advantageous properties such as improved transfer, delivery, tolerance, and the like.

A composition comprising one or more PIRAs for pharmaceutical use can include various diluents, fillers, salts, buffers, detergents (e.g., a nonionic detergent, such as Tween-80), stabilizers (e.g., sugars or protein-free amino acids), preservatives, tissue fixatives, solubilizers, and/or other materials suitable for inclusion in a composition for pharmaceutical use. Examples of suitable components also are described in, e.g., Berge et al., J. Pharm. Sci., 6661), 1-19 (1977); Wang and Hanson, J. Parenteral. Sci. Tech: 42, S4-S6 (1988); U.S. Pat. Nos. 6,165,779 and 6,225, 289; and other documents cited herein. Such a pharmaceutical composition also can include preservatives, antioxidants, or other additives known to those of skill in the art. Additional pharmaceutically acceptable carriers are known in the art and described in, e.g., Urquhart et al., Lancet, 16, 367 (1980), Lieberman et al., Pharmaceutical Dosage Forms-Disperse Systems (2nd ed., vol. 3, 1998); Ansel et al., Pharmaceutical Dosage Forms & Drug Delivery Systems (7th ed. 2000); Martindale, The Extra Pharmacopeia (31st edition), Remington's Pharmaceutical Sciences (16th-20th editions); The Pharmacological Basis Of Therapeutics, Goodman and Gilman, Eds. (9th ed.-1996); Wilson and Gisvolds' Textbook Of Organic Medicinal And Pharmaceutical Chemistry, Delgado and Remers, Eds. (10th ed.—1998), and U.S. Pat. Nos. 5,708,025 and 5,994,106. Principles of formulating pharmaceutically acceptable compositions also are described in, e.g., Platt, Clin. Lab Med., 7: 289-99 (1987), Aulton, Pharmaceutics: The Science Of Dosage Form Design, Churchill Livingstone (New York) (1988), Extemporaneous Oral Liquid Dosage Preparations, CSHP (1998), and “Drug Dosage,” J. Kans. Med. Soc., 70 (I), 30-32 (1969). Additional pharmaceutically acceptable carriers particularly suitable for administration of PIRA compositions and related compositions (e.g., compositions comprising PIRA-encoding nucleic acids or PIRA-encoding nucleic acid comprising vectors) are described in, for example, International Patent Application WO 98/32859.

PIRA compositions can be prepared with a carrier that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid, and combinations of any thereof, so as to provide such a composition. Methods for the preparation of such compositions are known. See, e.g., Sustained and Controlled Release Drug Delivery Systems, J. R. Robinson, ed., Marcel Dekker, Inc., New York, 1978.

In another aspect, compositions of the invention are formulated with excipients for oral administration, such as, for example, with an inert diluent or an assimilable edible carrier (specific oral administration formulations and methods are also separately described elsewhere herein). The PIRA compound (and other ingredients, if desired) may also be enclosed in a hard or soft shell gelatin capsule, compressed into tablets, or incorporated directly into the subject's diet. For oral therapeutic administration, the compounds may be incorporated with excipients and used in the form of ingestible tablets, buccal tablets, troches, capsules, elixirs, suspensions, syrups, wafers, and the like. To administer a compound of the invention by other than parenteral administration, it may be necessary to coat the compound with, or co-administer the compound with, a material to prevent its inactivation.

In another aspect, PIRA compositions are prepared as injectables, either as liquid solutions or suspensions, however, solid forms suitable for solution in, or suspension in, liquid prior to injection can also be prepared. The preparation can also be emulsified. The active therapeutic ingredient is often mixed with excipients that are pharmaceutically (i.e., physiologically) acceptable and compatible with the active ingredient. Suitable excipients are, for example, water, saline, dextrose, glycerol, ethanol, or the like and combinations thereof. In addition, if desired, the composition can contain minor amounts of auxiliary substances such as wetting or emulsifying agents, pH-buffering agents, which enhance the effectiveness of the active ingredient.

A PIRA alternatively can be formulated into a pharmaceutical composition as neutralized physiologically acceptable salt forms. Suitable salts include the acid addition salts (i.e., formed with the free amino groups of the peptide molecule) and which are formed with inorganic acids such as, for example, hydrochloric or phosphoric acids, or such organic acids as acetic, oxalic, tartaric, mandelic, and the like. Salts formed from the free carboxyl groups can also be derived from inorganic bases such as, for example, sodium, potassium, ammonium, calcium, or ferric hydroxides, and such organic bases as isopropylamine, trimethylamine, 2-ethylamino ethanol, histidine, procaine, and the like. In the case of combination compositions (discussed further herein), PIRAs can be coformulated with and/or coadministered with one or more additional therapeutic agents. Such combination therapies may require lower dosages of the PIRA and/or the co-administered agents, so as to avoid possible toxicities or complications associated with the various monotherapies.

In another facet, the invention provides PIRA-encoding nucleic acids (i.e., nucleic acids comprising one or more sequence that together or separately code for the expression of a PIRA in one or more suitable host cells). PIRA-encoding nucleic acids can have any suitable characteristics and comprise any suitable features. Thus, for example, a PIRA-encoding nucleic acid may be in the form of DNA, RNA, or a hybrid thereof, and may include nonnaturally-occurring bases or nucleotide analogues, replacement of sugar moieties, conjugation of additional molecules (e.g., uptake promoting molecules); inclusion of a modified backbone (e.g., a phosphothioate backbone that promotes stability of the nucleic acid), secondary structure-promoting sequences, or combinations of such features. A nucleic acid typically advantageously comprises features that promote desired expression, replication, and/or selection in target host cell(s). Examples of such features include an origin of replication component, a selection gene component, a promoter component, an enhancer element component, a polyadenylation sequence component, a termination component, and the like, numerous suitable examples of which are known.

In a further aspect, the invention provides vectors comprising one or more PIRA-encoding nucleic acids. A “vector” refers to a delivery vehicle that promotes the expression of a PIRA-encoding nucleic acid, the production of a PIRA peptide, the transfection/transformation of target cells, the replication of the PIRA-encoding nucleic acid, promotes stability of the nucleic acid, promotes detection of the nucleic acid and/or transformed/transfected cells, or otherwise imparts advantageous biological and/or physiochemical function to the PIRA-encoding nucleic acid. A vector in the context of this invention can be any suitable vector, including chromosomal, non-chromosomal, and synthetic nucleic acid vectors (a nucleic acid sequence comprising a suitable set of expression control elements). Examples of such vectors include derivatives of SV40, bacterial plasmids, phage DNA, baculovirus, yeast plasmids, vectors derived from combinations of plasmids and phage DNA, and viral nucleic acid (RNA or DNA) vectors. In one exemplary aspect, a PIRA-encoding nucleic acid is comprised in a naked DNA or RNA vector, including, for example, a linear expression element (as described in, e.g., Sykes and Johnston (1997) Nat Biotech 17: 355-59), a compacted nucleic acid vector (as described in, e.g., U.S. Pat. No. 6,077,835 and/or International Patent Application WO 00/70087), a plasmid vector such as pBR322, pUC 19/18, or pUC 118/119, a “midge” minimally-sized nucleic acid vector (as described in, e.g., Schakowski et al. (2001) Mol Ther 3: 793-800), or as a precipitated nucleic acid vector construct, such as a CaP0₄-precipitated construct (as described in, e.g., International Patent Application WO 00/46147, Benvenisty and Reshef (1986) Proc Natl Acad Sci USA 83: 9551-55, Wigler et al. (1978), Cell 14:725, and Coraro and Pearson (1981) Somatic Cell Genetics 7: 603). Such nucleic acid vectors and the usage thereof are well known in the art (see, e.g., U.S. Pat. Nos. 5,589,466 and 5,973,972). In one aspect, the invention provides a vector comprising a PIRA-encoding nucleic acid, wherein the vector is suitable for expression of the PIRA in a bacterial cell. Examples of such vectors include, for example, vectors which direct high level expression of fusion proteins that are readily purified (e.g., multifunctional E. coli cloning and expression vectors such as BLUESCRIPT (Stratagene), pIN vectors (Van Heeke & Schuster, J Biol Chem 264: 5503-5509 (1989); pET vectors (Novagen, Madison Wis.) ; and the like). A suitable PIRA expression vector also or alternatively can be, for example, a vector suitable for expression in a yeast system. Any vector suitable for expression in a yeast system can be employed. Suitable vectors for use in, e.g., Saccharomyces cerevisiae include, for example, vectors comprising constitutive or inducible promoters such as alpha factor, alcohol oxidase and PGH (reviewed in, e.g., Ausubel, supra, and Grant et al., Methods in Enzymol 153: 516-544 (1987)).

A nucleic acid and/or vector can also comprise a nucleic acid sequence encoding a secretion/localization sequence, which can target a polypeptide, such as a nascent polypeptide chain, to a desired cellular compartment, membrane, or organelle, or which directs polypeptide secretion to periplasmic space or into cell culture media. Such sequences are known in the art, and include secretion leader or signal peptides, organelle targeting sequences (e.g., nuclear localization sequences, ER retention signals, mitochondrial transit sequences, chloroplast transit sequences), membrane localization/anchor sequences (e.g., stop transfer sequences, GPI anchor sequences), and the like.

PIRA expression vectors can comprise or be associated with any suitable promoter, enhancer, and other expression-facilitating elements. Examples of such elements include strong expression promoters (e.g., a human CMV IE promoter/enhancer, an RSV promoter, SV40 promoter, SL3-3 promoter, MMTV promoter, or HIV LTR promoter), effective poly (A) termination sequences, an origin of replication for plasmid product in E. coli, an antibiotic resistance gene as a selectable marker, and/or a convenient cloning site (e.g., a polylinker). Nucleic acids also can comprise an inducible promoter as opposed to a constitutive promoter such as CMV IE (the skilled artisan will recognize that such terms are actually descriptors of a relative degree of gene expression under certain conditions). In one aspect, the invention provides a nucleic acid comprising a sequence encoding a PIRA which is operatively linked to a tissue specific promoter.

In another aspect, a PIRA-encoding nucleic acid is positioned in and/or delivered to the host cell or host animal via a viral vector. Any suitable viral vector can be used in this respect, and several are known in the art. A viral vector can comprise any number of viral polynucleotides, alone or in combination with one or more viral proteins, which facilitate delivery, replication, and/or expression of the nucleic acid of the invention in a desired host cell. The viral vector can be a polynucleotide comprising all or part of a viral genome, a viral protein/nucleic acid conjugate, a virus-like particle (VLP), a vector similar to those described in U.S. Pat. No. 5,849,586 and International Patent Application WO 97/04748, or an intact virus particle comprising viral nucleic acids and the nucleic acid of the invention. A viral particle viral vector can comprise a wild-type viral particle or a modified viral particle. The viral vector can be a vector which requires the presence of another vector or wild-type virus for replication and/or expression (i.e., a viral vector can be a helper-dependent virus), such as an adenoviral vector amplicon. Typically, such viral vectors consist essentially of a wild-type viral particle, or a viral particle modified in its protein and/or nucleic acid content to increase transgene capacity or aid in transfection and/or expression of the nucleic acid (examples of such vectors include the herpes virus/AAV amplicons). Typically, a viral vector is similar to and/or derived from a virus that normally infects humans. Suitable viral vector particles in this respect, include, for example, adenoviral vector particles (including any virus of or derived from a virus of the adenoviridae), adeno-associated viral vector particles (AAV vector particles) or other parvoviruses and parvoviral vector particles, papillomaviral vector particles, flaviviral vectors, alphaviral vectors, herpes viral vectors, pox virus vectors, retroviral vectors, including lentiviral vectors. Examples of such viruses and viral vectors are in, e.g., Fields et al. , eds. , VIROLOGY Raven Press, Ltd., New York (3^{rd} ed., 1996 and 4^{th} ed., 2001); ENCYCLOPEDIA OF VIROLOGY, R. G. Webster et al., eds., Academic Press (2nd ed., 1999); FUNDAMENTAL VIROLOGY, Fields et al., eds., Lippincott-Raven (3rd ed., 1995), Levine, “Viruses,” Scientific American Library No. 37 (1992), MEDICAL VIROLOGY, D. O. White et al., eds., Acad. Press (2nd ed. 1994), and INTRODUCTION To MODERN VIROLOGY, Dimock, N.J. et al., eds., Blackwell Scientific Publications, Ltd. (1994). Viral vectors that can be employed with polynucleotides and other nucleic acids of the invention and the methods described herein thus include, for example, adenoviral viral vectors; adeno-associated viral (AAV) vectors; papillomaviral vectors; herpes viral vectors; retroviral vectors, including lentiviral vectors, pox viral vectors (e.g., vaccinia virus vectors); and the like.

The invention also provides recombinant cells, such as yeast, bacterial, and mammalian cells (e.g., immortalized mammalian cells) comprising a PIRA-encoding nucleic acid, vector, or combination of either or both thereof. For example, in one exemplary aspect the invention provides a cell comprising a nucleic acid stably integrated into the cellular genome that comprises a sequence coding for expression of a PIRA of the invention. In another aspect, the invention provides a cell comprising a non-integrated nucleic acid, such as a plasmid, cosmid, phagemid, or linear expression element, which comprises a sequence coding for expression of a PIRA.

Nucleic acids, vectors, and cells can be used as surrogates for PIRA proteins of the invention in most of the inventive methods described herein, e.g., so as to “deliver” one or more PIRAs to a cell in culture or a host (as opposed to “administer” the PIRA by other methods described above (e.g., peptidic compound composition injection, inhalation, etc.)). Thus, for example, the invention provides a method of antagonizing IR in the cells of a mammal that comprises introducing a vector comprising a PIRA-encoding nucleic acid to suitable cells under conditions suitable for expression of the nucleic acid and production of a PIRA such that the expressed PIRA comes in contact with cells comprising IRs responsive thereto so as to antagonize the activity of such IRs.

The invention also provides transgenic organisms comprising PIRA-encoding nucleic acids or vectors comprising the same. Suitable transgenic organisms include mice, rats, chickens, plants, cows, goats, guinea pigs, monkeys, and other non-human primates. Transgenic animals can be produced by stable introduction of PIRA-encoding nucleic acids according to standard techniques.

Another aspect of the invention is embodied in new uses of PIRAs, PIRA compositions, and related molecules and compositions (e.g., PIRA-encoding nucleic acids, associated vectors, etc.).

In one aspect, the invention provides a method of treating a disease, disorder, or condition in a subject (e.g., a mammalian host, for example a household pet, livestock, or human patient) in which antagonism of the IR is beneficial comprising administering (or delivering, these terms generally being interchangeable herein, unless specifically stated) a therapeutically effective amount of a PIRA to the subject so as to treat the disease, disorder, or condition. Terms such as “treatment” refer to the delivery (e.g., administration) of an effective amount of a therapeutically active PIRA compound or composition of the invention with the purpose of preventing any symptoms or disease state to develop or with the purpose of easing, ameliorating, or eradicating (curing) such symptoms or disease states already developed. The term “treatment” is thus generally meant to include prophylactic treatment. However, it will be understood that therapeutic regimens and prophylactic regimens of the invention also can be considered separate and independent aspects of this invention. In another aspect, the invention provides for the use of PIRAs in the production of medicaments useful in the treatment of such disorders and conditions. As indicated above, PIRAs can be administered individually or in combination with other pharmacologically active agents. It will be understood that such combination therapy encompasses different therapeutic regimens, including, without limitation, administration of multiple agents together in a single dosage form or in distinct, individual dosage forms. If the agents are present in different dosage forms, administration may be simultaneous or near-simultaneous or may follow any predetermined regimen that encompasses administration of the different agents.

In one aspect, the invention provides a method of treating hyperinsulinemia in a subject comprising delivering an effective amount of a PIRA to the subject so as to treat the condition. In one aspect, the invention provides a method of treating a subject (e.g., a human patient) suffering from hyperinsulinemia that is characterized as having a blood insulin level that corresponds to at least about the 75^{th} percentile fasting insulin level (e.g., at least about the 80^{th} percentile, 85^{th} percentile, the 90^{th} percentile, the 95^{th} percentile, etc.) for the population group (e.g., a population of nonobese men with normal glucose tolerance and no diabetic history or medication use). In one aspect, the subject having hyperinsulinemia is defined as a human patient having fasting insulin levels of about 10 μU/mL (60 pmol/L) or greater, such as about 12 μU/mL (72 pmol/L) or greater (e.g., at least about 13 μU/mL (78 pmol/L) , at least about 14 μU/mL (84 pmol/L), at least about 15 μU/mL (90 pmol/L), at least about 16 μU/mL (96 pmol/L), at least about 17 μU/mL (102 pmol/L), at least about 18 μU/mL (108 pmol/L), at least about 19 μU/mL (114 pmol/L), at least about 20 μU/mL (120 pmol/L), or more than 20 μU/mL (120 pmol/L)).

In particular aspects, the invention provides methods of reducing the likelihood of the onset of disorders and symptoms related to hyperinsulinemia in a subject, such as reducing anxiety, abnormal hunger, abnormal fatigue, overeating, psychiatric symptoms associated with low blood sugar, and/or hypoglycemia (including hypoglycemia-related seizure, coma, and death). The inventive methods can be applied to treat various types of persistent hyperinsulinemia conditions, such as nesidioblastosis (KATP-HI Diffuse Disease, KATP-HI Focal Disease, or “PHHI”), GDH-HI (Hyperinsulinism/Hyperammonaemia Syndrome (HI/HA), leucine-sensitive hypoglycemia, or diazoxide-sensitive hypoglycemia), islet cell dysregulation syndrome, idiopathic hypoglycemia of infancy, Persistent Hyperinsulinemic Hypoglycemia of Infancy (PHHI), Congenital Hyperinsulinism, and insulinoma. The inventive method of administering or otherwise delivering a PIRA to treat or prevent hyperinsulinemia can in anther aspect be applied to subjects suffering from or being at imminent risk of developing polycystic ovary syndrome (PCOS), which frequently is associated with hyperinsulinemia.

In another particular aspect, the invention provides a method of reducing the likelihood of developing and/or delaying the onset time of (and/or eventual severity of) hyperinsulinemia-associated hypoglycemia in a subject by administering an effective amount of a PIRA to the subject.

In yet another particular aspect, the invention provides a method of treating (providing therapy for and/or preventing) neonatal hypoglycemia (or other “transient hyperinsulinemia”) brought upon by hyperinsulinemia, comprising delivering to the infant subject an effective amount of a PIRA so as to treat the condition or to prevent, reduce likelihood of, or delay the onset of associated conditions such as seizures and neurologic sequelae.

In another aspect, the invention provides a method of reducing the severity of hypoglycemia in a patient suffering from a hyperinsulinemia-related condition.

In yet another aspect, the invention provides a method of improving or maintaining the health of a patient diagnosed as having or being at substantial risk of developing insulinomas comprising delivering to the host an effective amount (either a therapeutically effective or prophylactically effective amount) of a PIRA.

In yet another aspect, the invention provides a method for maintaining or improving the health in an individual (and/or modulating IR activity in an individual), such as an individual having characteristics associated with high risk of hyperinsulinemia (e.g., a known familial history of hyperinsulinemia-related conditions or being a member of a population group with a relatively high occurrence of hyperinsulinemia, such as in men of ethnic Japanese origin), comprising administering (or otherwise delivering) to the individual a physiologically effective amount (e.g., a prophylactically effective amount) of a PIRA.

In another aspect, the invention provides a method for antagonizing IR activity in a cell (e.g., in an appropriate cell population in vitro or in a suitable vertebrate (e.g., chordate) host) comprising delivering to the cells a physiologically effective amount of a PIRA.

In still another aspect, the invention provides a method of reducing the effect of circulating insulin in a subject comprising delivering a physiologically effective amount of a PIRA to the subject.

In another aspect, the invention relates to a therapy for hyperinsulinemia or hyperinsulinemia-related condition (such as hyperinsulinemia-related hypoglycemia) wherein the therapy comprises delivering an effective amount of a PIRA in combination with a relatively reduced amount of secondary agent (e.g., glucagon), as compared to the amount that would be used without the PIRA to achieve a similar therapeutic effect.

In a further aspect, the invention provides a method for lowering blood glucose levels in a subject comprising delivering to the subject a physiologically effective amount of a PIRA.

In yet another aspect, the invention provides a method of reducing the effects of insulin in a subject comprising administering to the subject a physiologically effective amount of a PIRA.

In another aspect, the invention provides a method for increasing insulin secretion in a subject comprising administering or otherwise delivering a physiologically effective amount of a PIRA to the subject so as to increase insulin secretion therein. Why not wishing to be bound by any particular theory, experimental evidence associated with administration of PIRA in test mammal suggests that insulin secretion is increased, presumably due to a shut off of an IR-mediated feedback system therein. In another aspect, the invention provides a method of treating Type I diabetes, Type II diabetes, obesity, impaired glucose tolerance, and/or impaired fasting glucose tolerance, the method comprising administering (or otherwise delivering) to a subject (e.g., a human patient diagnosed as being in need thereof) a prophylactically or therapeutically effective amount of a PIRA, optionally in combination with one or more other therapeutically active agents suitable for the treatment of the disorder to be treated (e.g., an anti-diabetic agent). In another aspect, the invention relates to the use of a PIRA (or related compound/composition—such as a nucleic acid encoding a PIRA) in the manufacture of a medicament for the treatment of Type I diabetes, Type II diabetes, obesity, impaired glucose tolerance, and/or impaired fasting glucose tolerance.

In another aspect, the invention relates to the preparation of medicaments for any of the above-described therapeutic regimens or other therapeutic purposes described herein by use of one or more PIRAs, optionally with other secondary therapeutic agents described herein. Thus, for example, in a particular aspect, the invention provides for the use of a PIRA in the preparation of a medicament for the treatment of hyperinsulinemia.

As indicated elsewhere herein, PIRA(s) can be, where appropriate, co-administered with or administered in association with secondary agents, which may include, for example, one or more anti-hyperinsulinism (e.g., insulin production-reducing) agents and/or anti-hypoglycemia agents such as diazoxide, octreotide, nifedipine, sandostatin, glucagon, or combinations of any or all thereof, in the practice of the therapeutic methods described herein (compositions comprising such combinations of agents also are a feature of the invention). PIRA administration also can be provided in association with surgical procedures used to treat hyperinsulinemia-related conditions, such as pancreatectomy.

In general , PIRAs can be delivered by any suitable manner, such as by expression from a nucleic acid that codes for production of the PIRA in target host cells (e.g., by expression from a PIRA-encoding nucleic acid under the control of an inducible promoter and comprised in a suitable gene transfer vector, such as a targeted and replication-deficient gene transfer vector) in the practice of the inventive methods described herein. Typically, PIRAs are delivered by direct administration of the PIRA or PIRA composition to a recipient host. Thus, PIRAs and PIRA compositions may be administered as pharmaceutical compositions comprising standard carriers known in the art for delivering proteins and peptides and/or delivered by gene therapy. In general, and where appropriate, the terms administration and delivery should be construed as providing support for one another herein (e.g., it should generally be recognized that PIRA-encoding nucleic acids can be used to deliver naked PIRAs to target host tissues as an alternative to direct administration of PIRA proteins), although it also should be recognized that each such method is a unique aspect of the invention with respect to any particular molecule and that some molecules (e.g., conjugated PIRAs comprising degradation-resistant organic moieties) are amenable to only certain forms of delivery/administration. Methods for the administration of proteins, nucleic acids, and related compositions (e.g., vectors and host cells), are well known and, accordingly, only briefly described herein.

PIRA compositions, related compositions, and combination compositions can be administered via any suitable route, such as an oral, mucosal, buccal, intranasal, inhalable, intravenous, subcutaneous, intramuscular, parenteral, or topical route. Such proteins may also be administered continuously via a minipump or other suitable device.

A PIRA or other PIRA generally will be administered for as long as the disease condition is present, provided that the protein causes the condition to stop worsening or to improve. The PIRA will generally be administered as part of a pharmaceutically acceptable composition, e.g., as described in detail elsewhere herein.

In general, a PIRA or related compound/composition may be administered by any suitable route, but typically is administered parenterally in dosage unit formulations containing conventional pharmaceutically acceptable carriers, adjuvants, and the like (stabilizers, disintegrating agents, anti-oxidants, etc.). The term “parenteral” as used herein includes, subcutaneous, intravenous, intraarterial, intramuscular, intrasternal, intratendinous, intraspinal, intracranial, intrathoracic, infusion techniques and intraperitoneal delivery. Thus, in one aspect, a PIRA composition is administered intravenously or subcutaneously, in practicing therapeutic methods of the invention. Routes of injection also include injection into the muscle (intramuscular IM); injection under the skin (subcutaneous (s.c.)); injection into a vein (intravenous (IV)); injection into the abdominal cavity (intraperitoneal (IP)); and other delivery into/through the skin (intradermal delivery, usually by multiple injections, which may include biolistic injections).

In one aspect the invention provides a method of modulating IR activity in a host comprising administering a pharmaceutical composition that includes, in admixture, a pharmaceutically (i.e., physiologically) acceptable carrier, excipient, or diluent, and one or more IR agonist PIRAs as an active agent component (which may be further combined with secondary active agents as described elsewhere).

Pharmaceutical compositions of the invention can be administered systemically by oral or parenteral routes. Non-limiting parenteral routes of administration include subcutaneous, intramuscular, intraperitoneal, intravenous, transdermal, inhalation, intranasal, intra-arterial, intrathecal, enteral, sublingual, or rectal. Due to the labile nature of typical amino acid sequences, parenteral administration may be advantageous. Advantageous modes of administration include, e.g., aerosols for nasal or bronchial absorption; suspensions for intravenous, intramuscular, intrasternal or subcutaneous, injection; and compounds for oral administration.

Intravenous administration, for example, can be performed by injection of a unit dose. The term “unit dose” when used in reference to a pharmaceutical composition of the present invention refers to physically discrete units suitable as unitary dosage for humans, each unit containing a predetermined quantity of active material calculated to produce the desired therapeutic effect in association with the required diluent; i.e., liquid used to dilute a concentrated or pure substance (either liquid or solid), making that substance the correct (diluted) concentration for use. For injectable administration, the composition is in sterile solution or suspension or may be emulsified in pharmaceutically- and physiologically-acceptable aqueous or oleaginous vehicles, which may contain preservatives, stabilizers, and material for rendering the solution or suspension isotonic with body fluids (i.e., blood) of the recipient.

Excipients suitable for use with typical formulations include water, phosphate buffered saline, aqueous sodium chloride solution, dextrose, glycerol, dilute ethanol, and the like, and mixtures thereof. Illustrative stabilizers are polyethylene glycol, proteins, saccharides, amino acids, inorganic acids, and organic acids, which may be used either on their own or as admixtures. The amounts or quantities, as well as routes of administration, used are determined on an individual basis, and correspond to the amounts used in similar types of applications or indications known to those of skill in the art.

Pharmaceutical compositions can typically be administered in a manner compatible with the dosage formulation, and in a therapeutically effective amount. The quantity to be administered depends on the subject to be treated and the degree of IR modulation to desired (e.g., the desired therapeutic or prophylactic outcome or effect). Precise amounts of active ingredient required to be administered depend on the judgment of the practitioner and are specific for each individual. However, suitable dosages may range from about 1-200 nmol (e.g., about 10 to 200—such as about 1-100, such as about 10-100) nmol active peptide per kilogram body weight of individual per day and depend on the route of administration. Suitable regimes for initial administration and booster shots are also variable, but are typified by an initial administration followed by repeated doses at one or more hour intervals by a subsequent injection or other administration. Alternatively, continuous intravenous infusions sufficient to maintain picomolar concentrations (e.g., approximately 1 pM to approximately 10 nM) in the blood are contemplated.

In another particular aspect, a PIRA or a PIRA composition is delivered by an injectable pump in a liquid or other suitable formulation for use with such devices. PIRAs also can be administered by pens, such as are currently used to deliver insulin products. The use of transdermal patches (e.g., a drug in matrix patch) also can be used to deliver PIRAs (e.g., by passive delivery or via iontophoretic delivery).

Further guidance in preparing pharmaceutical formulations can be found in, e.g., Gilman et al. (eds), 1990, Goodman and Gilman's: The Pharmacological Basis of Therapeutics, 8th ed., Pergamon Press; and Remington's Pharmaceutical Sciences, 17th ed., 1990, Mack Publishing Co., Easton, Pa.; Avis et al. (eds), 1993, Pharmaceutical Dosage Forms: Parenteral Medications, Dekker, New York; Lieberman et aL (eds), 1990, Pharmaceutical Dosage Forms: Disperse Systems, Dekker, New York.

To better illustrate particular aspects, a detailed discussion of dosage principles is further provided here.

In one aspect, the inventive methods comprise administering or otherwise delivering two different PIRAs over a period of one month, the beginning of the therapy involving the second PIRA starting about 1-3 weeks (e.g., about 10 days) after the first delivery of the first PIRA or at any time when a significant immune response to the first PIRA develops in the host, such that the continued use of the first PIRA has become detrimental to the patient.

A particularly advantageous aspect of the invention is embodied in a pharmaceutically acceptable composition comprising a therapeutically and/or prophylactically effective amount of one or more digestive enzyme stabilized PIRAs (comprising one or more unusual degradation-resistant amino acid residues and/or degradation resistant moieties as described above) formulated for oral administration and the use of such a composition in the modulation of IR activity (e.g., in the context of treating diabetes or a related condition, such as IR-modulated metabolic disorder). The relatively small size of typical PIRAs and simple structure (e.g., a single chain of about 40 amino acid residues in the case of many PIRAs) in and of itself is believed to aid in the oral delivery of such proteins as compared to larger proteins such as antibodies (which may be hundreds of amino acids in size, spread across at least two amino acid chains). The addition of N- and/or C-terminal blocking modifications (particularly, e.g., acetylation (or other acylation) and amidation, respectively) are believed to increase the ability of such molecules to be delivered orally. The inclusion of degradation-resistant unusual amino acid residues and/or organic moieties also or alternatively is believed to significantly increase the ability of such peptides to be delivered effectively by an oral route as compared to presently known forms of insulins and insulin analogs. PIRAs comprising a combination of such features are believed to be particularly advantageous for oral delivery suitable therapeutic agents. In one aspect, the invention provides PIRA oral formulations that may exhibit at least about 1%, at least about 5%, at least about 10%, at least about 15%, at least about 20% or more relative bioavailability upon oral administration as compared to parenteral injection. The inherent oral delivery capacity of PIRAs can be enhanced by formulating the PIRAs with oral delivery enhancing compositions using methods known in the art and that have been demonstrated to be effective in enhancing the oral delivery and availability of small peptides. Typically, oral formulations seek to inhibit or modulate proteolytic activity that degrades the peptide; enhance paracellular and/or transcellular transport of the peptide; improve peptide penetration through the mucus barrier (particularly in the case of fast-dissolving forms and aerosol-delivered or spray-delivered orally administered forms); and/or increase the half-life of the peptide in circulation (particularly for peptides that require a sustained presence for therapeutic efficacy). Devices can assist in such delivery. For example, PIRA compositions can be formulated for delivery by an aerosol spray device that allows delivery of the composition to the buccal mucosa and oropharynx region, wherein absorption of the formulation can occur. Examples of such devices, used for delivery of small peptides, such as insulin, are known in the art.

In one aspect, the invention provides a PIRA oral formulation composition wherein a PIRA is conjugated to a carrier molecule or encapsulated to improve stability of the PIRA as compared to the stability of the PIRA without the stabilizing conjugate or encapsulation materials(s). PEG conjugates and example of a typically stabilizing and delivery enhancing conjugate material. PIRAs can be, for example, conjugated to a PEG-based amphiphilic oligomer that increases GI absorption and/or reduces proteolytic degradation of the PIRA. In another exemplary aspect, calcium phosphate-PEG-insulin-casein (CAPIC) particles encasing PIRA compositions are used as an oral delivery form (microparticle and nanoparticle forms are further described elsewhere in this section). In one aspect, a PIRA is conjugated to a delivery agent or carrier that facilitates passive transcellular transport. Desirably, such a carrier or agent is engineered so as to disassociate from the PIRA in circulation (e.g., upon reaching a certain level of exposure to low pH conditions).

In another aspect, a PIRA is formulated in an enteric-coated microcapsule or table containing one or more oral delivery facilitating excipients, such as sodium cholate and/or a trypsin inhibitor. In yet a further aspect, a PIRA oral formulation is provided that comprises an effective amount of a detergent component that increases the solubility of the peptide, decreases interactions with intestinal mucus, and/or enhances paracellular transport. In still another aspect, a PIRA is conjugated to one or more (typically several) low molecular weight (LMW) polymer conjugates that facilitate oral delivery by, e.g., adding resistance to enzymatic degradation with respect to related/similar naked PIRAs and/or allowing better gastrointestinal transport (e.g., improved diffusion through both water and fatty portions of cells and tissues that make up barriers to absorption along the gastrointestinal pathway and into the bloodstream). Compositions, methods, and relevant principles for the construction of oral delivery-enhancing small peptide conjugates are provided in, e.g., U.S. Pat. Nos. 5,359,030; 5,438,040; 5,681,811; and 6,309,633. In another aspect, the invention provides an oral delivery PIRA formulation that comprises an amino acid-based capsule system, which promotes intestinal lining passage and inhibits enzymatic degradation of the PIRA.

In still another aspect, the invention provides a PIRA oral delivery formulation that comprises a lipid or liposome encapsulation of a PIRA or PIRA composition, which promotes transmission through epithelial barrier(s) and/or protects the PIRA from enzymatic degradation. Desirably, such lipid formulations promote significant absorption of the pharmaceutical composition by the oral mucosa, thereby avoiding the “first pass” effect. In another facet, the invention provides PIRA oral delivery formulations wherein a PIRA is contained within microparticles, which are administered directly or inserted in capsules, packets and the like for direct oral administration or administration by an oral delivery device (other oral delivery forms described herein generally can be delivered to a host by such methods as well). In a particular aspect, the invention provides a PIRA oral delivery formulation composed of alginate microspheres. Desirably, the alginate is a naturally occurring alginate that is classified as generally regarded as safe by the US FDA and optionally also induces or promotes a protective effect on the mucous membrane of the upper gastrointestinal tract. In another aspect, a coated crystal formulation is provided. Extrusion spheronization is a technology that permits high concentrations of active substances to be included in high drug concentration pellets and that may be applicable to PIRA formulations.

In another aspect, a PIRA composition is presented as a dry syrup suspension of coated particles in a bottle or other container (e.g., a unit dose container) for liquid oral administration.

In general, formulations described herein can be sustained-release formulated, taste-masked or entero-coated compositions.

In another aspect, the invention provides a semi-solid matrix system in a relatively hard gelatin capsule for oral administration. Typically, such capsules further comprise a delivery promoting agent, such as a lipidic peptide delivery system. Soft pellet tablets comprising coated microparticles also are provided by the invention. Matrix tablets, which can be hydro-inert and/or lipo-inert, are another potentially suitable oral delivery form. Typically, such tables comprise coated microparticles of PIRA compositions and optionally peptidase inhibitors and/or penetration enhancers or other suitable excipients. Suitable penetration enhancers can include surfactants, fatty acids, bile salts, citrates, and chelators (e.g., EDTA), although other suitable penetration enhancers also can be included in these compositions or in other oral delivery forms described herein. For example, cyclodextrins may be used to enhance penetration of PIRA microparticles, conjugates, or other drug forms.

In an additional facet, the invention provides a PIRA composition comprising a bioadhesive polymer or other bioadhesive material, which typically facilitates association with the GI tract. Examples of such polymers include polycarbophil and chitosan. As already mentioned, carrier systems, such as nanoparticles, microspheres, liposomes, and the like (e.g., small unilamellar vesicles (SUVs); albumin-containing nanoparticles; methylmethacrylate-containing nanoparticles; and albumin-containing microspheres (e.g., albumin/iron oxide magnetic and targetable microspheres or other targetable microparticle/nanoparticle formulations)) also or alternatively can be used to promote oral administration of a PIRA composition to a patient. Such formulations may be engineered to enhance absorption from the various regions of the GI tract and/or prevent degradation of the PIRA composition.

Emulsions and microemulsions also can be used as oral delivery formulations for PIRAs. For example, a water-in-oil emulsion comprising a hydrophobic phase comprising oleic acid, gadoleic acid, erucic acid, linoleic acid, linolenic acid, ricinoleic acid, arachidonic acid, glyceryl esters of such acids, oleyl alcohol, d-alpha-tocopherol polyethylene glycol succinate, combinations of any thereof, or similar molecules; a discontinuous aqueous hydrophilic phase; and at least one surfactant (e.g., poloxamer 124, a polyglycolized glyceride, sorbitan laurate, polyoxyethylene sorbitan monooleate, or similar surfactant can be included in such an emulsion and related pre-emulsion concentrate) for dispersing the hydrophilic phase (the hydrophobic phase typically forming about 5-10 wt. % of the emulsion) comprising the PIRA composition, which may include an alcohol, salt solution, etc.) in the hydrophobic phase (which typically is present in about 65-80 wt. % in the emulsion) as a water-in-oil emulsion may be useful in promoting oral delivery of PIRA compositions. Emulsions can be coated in enteric coating materials, which may be soluble in an acidic aqueous environment as mentioned with respect to other coating materials suitable for the oral delivery formulations of the invention.

In another aspect, the invention provides an oral delivery formulation comprising a PIRA associated with a thiolated polymer drug carrier matrix or polymer. An example of such a polymer is 2-Iminothiolane. In one aspect, such a polymer is covalently linked to chitosan to form a chitosan conjugate. Optionally, enzyme inhibitors (e.g., Bowman-Birk-Inhibitor and/or elastatinal) can be conjugated to the chitosan component of the conjugate. Also or alternatively, a permeation mediator can be included with the PIRA composition in tablets formed from such a chitosan. Immobilization of thiol groups on the polymer may enhance the mucoadhesive/cohesive properties of such formulations.

An encapsulation coat can include different combinations of pharmaceutical active ingredients, such as hydrophilic surfactants, lipophilic surfactants, and triglycerides. Sustained release oral delivery systems and/or enteric coatings for orally administered dosage forms are also contemplated such as those described in U.S. Pat. No. 4,704,295 issued Nov. 3, 1987, U.S. Pat. No. 4,556,552 issued Dec. 3, 1985, U.S. Pat. No. 4,309,404 issued Jan. 5, 1982 and U.S. Pat. No. 4,309,406 issued Jan. 5, 1982. Additional examples of solid carriers include bentonite, silica, and the like. Additional exemplary materials are described elsewhere herein.

In a further aspect, PIRA oral delivery forms comprising hydrogels are provided. PIRAs can be incorporated and orally delivered in hydrogels of poly(methacrylic acid-g-ethylene glycol), for example. PIRAs also can be complexed with hydrogels. pH-responsive complexation hydrogels, such as hydrogels containing pendent glucose (P(MAA-co-MEG)) or grafted LMW (e.g., about 200 MW) PEG chains (P(MAA-g-EG)), may be advantageously used as PIRA oral delivery agents.

Nanospheres, microspheres, and other nanoparticles/microparticles can be formed from crosslinked networks of methacrylic acid and/or acrylic acid grafted with PEG(s) may be useful oral delivery formulations. Methods for entrapping drug product in such nanospheres at low pH (e.g., about 3), but as releasable agents at physiological pH (e.g., about 7) are known in the art. Additional exemplary microparticle formulations for naked and conjugated small peptide delivery and related methods and principles are set forth in, e.g., U.S. Pat. No. 6,191,105.

An oral delivery formulation also can be presented as an inhalable composition selected from the group of aerosol, sprays and dry powders. Such pharmaceutical formulations of the present invention may be administered in the form of an aerosol spray using for example, a nebulizer such as those described in U.S. Pat. No. 4,624,251 issued Nov. 25, 1986; U.S. Pat. No. 3,703,173 issued Nov. 21, 1972; U.S. Pat. No. 3,561,444 issued Feb. 9, 1971 and U.S. Pat. No.4,635,627 issued Jan. 13, 1971. Other systems of aerosol delivery, such as the pressurized metered does inhaler (MDI) and the dry powder inhaler as disclosed in Newman, S. P. in Aerosols and the Lung, Clarke, S. W. and Davia, D. eds. pp.197-224, Butterworths, London, England, 1984, can be used when practicing the present invention.

In a further aspect, a PIRA composition is formulated as a mucoadhesive intestinal patch designed to deliver therapeutic doses of PIRA(s) into the systemic circulation of a patient. Such intestinal patches are thought to localize the associated PIRA near the mucosa and protect it from proteolytic degradation. Secure adhesion of such patches to the intestine has been demonstrated with insulin formulations and such patches have been shown to be effective for peptide drug delivery.

The oral delivery formulations described herein can be applied to the novel PIRAs specifically described herein, variants thereof (as described herein), PIRAs derived from the PIRAs described in the prior patent documents (as modified by one or more of the various aspects of this invention), or to even unmodified PIRAs described in the prior patent documents, provided that inclusion of such unmodified and previously characterized PIRAs in such compositions results in an increase in effectiveness in oral administration. The use of such compositions in the various methods of the invention is another facet of this invention.

In another aspect, the invention provides formulations for pulmonary delivery of PIRAs or PIRA compositions (e.g., compositions comprising combinations of PIRAs and/or PIRAs with secondary agents). PIRAs can be directly administered to the lungs or administered in standard pharmaceutical formulations to the lungs (due to the above-described advantageous characteristics of such molecules for these forms of delivery), but, more typically, are administered in formulations engineered for pulmonary delivery, such as in particles that can be delivered as an aerosol for inhalation. PIRA compositions can be, for example, prepared as dry powders for administration by a dry powder (and stored in a suitable composition prior to delivery such as a blister pack) inhaler. The particle size of the particles in the dry formulation for such a system typically is less than about 5 μm in diameter and such particles typically are about 90% or more pure for drug composition. Such compositions can be prepared through known “glass stabilization techniques.” Alternatively, PIRA compositions can be formulated in aqueous compositions (also optionally stored in blister packs) and administered by an aqueous mist inhaler (e.g., a microprocessor controlled aqueous mist inhaler engineered to ensure proper dosing). Such devices can provide an increase in delivery of at least about 5-fold, such as about 10-fold, over a conventional nebulizer. Nebulizers, metered-dose inhalers (MDIs) and dry-powder inhalers (DPIs) can be useful in delivering such formulations. A number of such devices and similar devices have been developed for pulmonary delivery of peptide drugs. Formulations also can be engineered for long IR modulation activity upon delivery, such as, in the case of particle drugs, by using enhancing agents and/or by employing long action-promoting particle features (e.g., porous particles comprising large quantities, such as at least about 50% poly(lactic acid-co-glycolic acid); dry particles with a small aerodynamic size (e.g., about 1-3 μm), low density (e.g., about 0.1 μm/ml or less), and large geometric particle size (e.g., about 10-20 μm).

PIRA compositions may comprise PIRA derivatives, such as low molecular weight PEGylated PIRAs, that enhance the pulmonary delivery of such molecules. In another aspect, PIRA compositions are delivered to the lungs in the form of PEG particles, calcium phosphate (CAP) particles, or PEG-CAP particles (typically in suspension), which particles can be prepared using standard techniques (e.g., controlled precipitation techniques). Such particle compositions can be specifically delivered to the lungs by, e.g., intratracheal instillation and/or spray instillation. Such particles can also be optionally associated with one or more caseins (e.g., as a coating for PEG, CAP, or PEG-CAP particles—formed by adding the particles to a solution of caseins and permitting the caseins to aggregate around and/or complex with the particles). Similar compositions also can be useful for oral delivery forms or nasal delivery forms (e.g., oral spray formulations) and such molecules can be used in other forms (e.g., hydrogels).

A number of strategies, compositions, and devices for pulmonary and oral delivery of small peptides, such as insulin, have been developed and described in the art, that may also be applied to the PIRA and PIRA compositions of the invention (e.g., by modifying the PIRA similar to the insulin molecule described therein; formulating a PIRA composition similar to the insulin formulation described therein; administering the PIRA using methods and/or devices similar to those described therein; etc.). Examples of such methods, principles, delivery devices, and similar compositions that may be useful in preparation of such formulations are described in, e.g., Garcia-Contreras et al., AAPS PharmSci 2003; 5(2) Article 9 (2003); Steiner et al., Exp Clin Endocrinol Diabetes. January 2002; 110(1):17-21; Pfutzner et al., Diabetes Technol Ther. 2002; 4(5):589-94; Gonda, I. et al.: Journal of Controlled Release 1998; 53: 269-274; Schuster, J. et al.: Pharmaceutical Research 1997; 14(3): 354-357; Farr, S. J. et al., Interpharm Press Inc., Buffalo Grove, Ill. 1996, pp. 175-184; Thippawong, J. et al., Diabetes Technology & Therapeutics 2002; 4(4): 499-504; Sangwan, S. et al., Journal of Aerosol Medicine. 2001 ;14(2):185-195, Mudumba S. et al., Respiratory Drug Delivery VII. Dalby R. N. et al (eds) Serentec Press, Raliegh, N.C. 2000. pp. 329-332; Brinda, Curr Opin Investig Drugs. May 2002; 3(5): 758-62; Brunner, G. A. et al., Diabetologia 1991; 44: 305-308; U.S. patent applications 20040096401; 20040089290; 20030216542; 20030148925; 20030113273; 20020046750; 20010039260; 20030150446; and U.S. Pat. Nos. 6,635,617; 6,518,239; 6,349,719; 6,335,316; 6,098,615; 6,024,090; 5,672,581; 5,915,378; 5,970,240; and 5,813,358.

### Other Applications

PIRAs may be applied in the investigation of any chemical or biochemical process involving the insulin receptor. The ability of certain PIRAs to block the insulin receptor from binding insulin can allow an investigator to determine the importance of the insulin receptor in both in vivo and in vitro processes involving insulin signaling or insulin clearance and/or degradation.

One example of such applications for PIRAs is in quantifying the extent of receptor-mediated degradation of insulin or insulin analogs in a mammal. The method comprises measuring the steady state plasma concentration of insulin or an insulin analog upon continuous infusion, either with or without administration of a PIRA by injection or infusion. From the ratio of steady state concentrations with and without PIRA administration, the contribution of receptor mediated degradation or clearance can be estimated.

Another example of such a method is embodied in quantifying the contribution of the insulin receptor to cellular signaling processes elicited by insulin or insulin analogs in the presence of other related cell surface receptors, such as e.g. the IGF1 receptor. The method comprises performing the effector assay in question with or without the addition of an appropriate concentration of PIRA to block the effects elicited through activation of the insulin receptor. The difference between results obtained with and without the addition of PIRA reflects the contribution of insulin receptor mediated signaling.

Yet another example of such a technique is administering a PIRA selectively to a tissue (e.g., the brain) (e.g., by localized administration or by expression from tissue specific targeted vectors, optionally wherein the PIRA-encoding sequence is under the control of a tissue-specific promoter) to determine the effect of blocking insulin receptor signaling in that particular tissue. Such a method may comprise delivering a PIRA that blocks insulin binding to a tissue under conditions wherein the effect of the PIRA is localized to the issue (e.g., using techniques such as those previously mentioned in the case of an in vivo study or by taking a tissue sample in the case of an in vitro study) in the presence of insulin and measuring known and desired insulin:insulin receptor reaction parameters (such as insulin-associated IR signaling).

Still another analytical method involving PIRAs is embodied in the identification of molecules that compete with PIRAs for binding to the IR, as a means of identifying additional IR binding agents and modulators (antagonists or agonists). Such methods involving competition-type assays wherein one or more PIRAs and one or more test compounds are added to a medium comprising an IR and assessing the ability of the test compound or test compounds to bind and modulate the IR in the presence of the PIRA(s).

In yet another example, a PIRA can be administered (or otherwise delivered) to a mammal (e.g., a suitable model animal for a human diabetes disease state) for an extended period of time so as to induce insulin resistance or diabetes, thus providing a novel and useful in vivo model for investigations involving diseases, including but not limited to diabetes and obesity.

### Exemplary Experimental Methods And Data

The following experimental methods serves to further illustrate inventive methods described herein but, of course, should not be construed as in any way limiting its scope.

### EXAMPLE 1

### PIRA Antagonist Assay

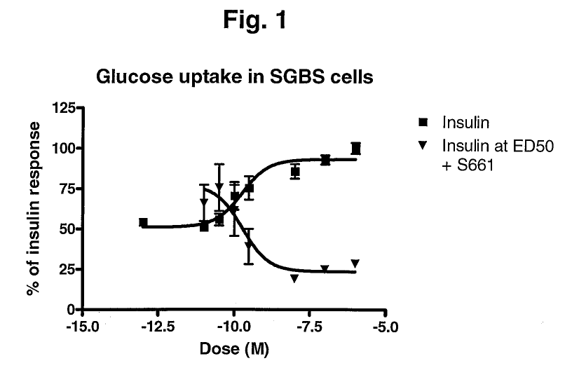

In order to identify an insulin receptor binding peptide as an antagonist (PIRA), a candidate peptide can be tested in a glucose uptake assay in e.g. SGBS cells (a human adipocyte cell line) or primary rodent adipocytes. Briefly, the cells are incubated with various concentrations of insulin and subsequently ¹⁴C-glucose uptake is measured. To test a peptide for antagonist properties, a submaximally stimulating concentration of insulin (e.g., resulting in half maximal insulin response) is added and also a dilution series of the peptide to be tested. If the peptide is a PIRA, the insulin induced response is reduced to or below the baseline response with increasing concentration of PIRA and the potency of the PIRA can be estimated from the concentration of PIRA necessary to observe the reduction in response. An example of such a potency determination of PIRA S661 is shown in FIG. 1. Such an assay can be applied to other candidate PIRAs as a method of identifying PIRAs and PIRAs exhibiting such properties in such an assay can be considered another feature of the invention.

### EXAMPLE 2

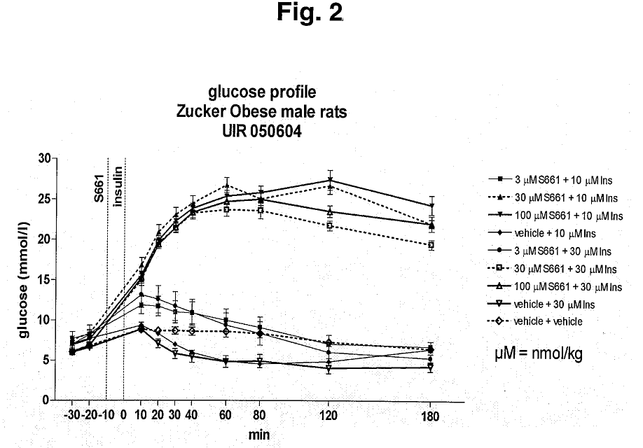

Nine groups of 5 or 6 male, 16 week old Zucker Obese rats (Charles River) with an average weight of about 580 gram were kept on ad lib Purina 5008 and ad lib drinking water and fasted overnight for the experiment. The glucose level was tested at 20 minutes and 10 minutes prior to administration of the PIRA designated S661 (described above) Intravenous (i.v.) administration of different doses of S661 in PBS or vehicle was followed by (10 minutes after administration of S661) intraperitoneal (i.p.) administration of different amounts of human insulin in PBS or vehicle. Blood glucose levels were measured at 10, 20, 30, 40, 60, 80, 120, and 180 minutes after injection of insulin. The results of these experiments are shown in FIG. 2.

As can be seen in FIG. 2, in rats in which S661 was administered, blood glucose levels were significantly higher than in cases where either insulin alone or only vehicle was administered (3 nmol/kg S661, 30 nmol/kg insulin resulted in a slightly higher glucose levels as compared to control and significantly higher levels than insulin alone; in all other cases the difference in blood glucose levels are significantly different where S661 was administered).

This experiment demonstrates that PIRAs, such as S661, are effective in reducing the effects of insulin in mammalian subjects.

### EXAMPLE 3

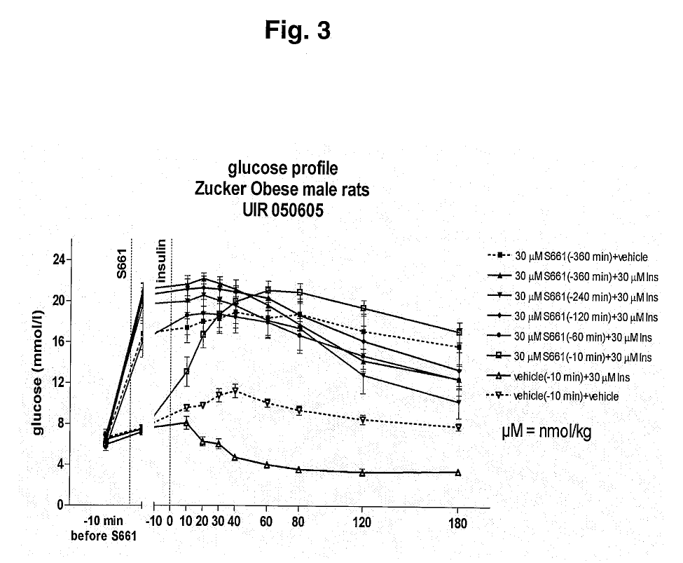

Eight groups of 5 to 7 male, 15 week old Zucker Obese rats (Charles River) with an average weight of about 550 gram were kept on ad lib Purina 5008 and ad lib drinking water and fasted overnight for the experiment. PIRA S661 in PBS was administered i.v. at 30 nmol/kg at various times before the i.p. administration of 30 nmol/kg human insulin in PBS. The results of these experiments are shown in FIG. 3. Blood glucose measurements over time showed that PIRA S661 is still an effective antagonist 6 hours after administration.

### EXAMPLE 4

To test the selectivity of PIRA S661 for the insulin receptor versus a related receptor, the IGF1 receptor, competition binding assays were carried out using both receptors and their cognate ligands in ¹²⁵I-labeled form. Whereas S661 had an affinity for the insulin receptor of 48% of that of human insulin itself, its affinity for the IGF1 receptor was too low to be measured, i.e. at least 200.000 fold lower than that of IGF1. These results indicate that PIRA S661 would not interfere with the signaling of IGF1 through the IGF1 -receptor in any way.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention are to be construed to cover both the singular and the plural, unless otherwise indicated herein or clearly contradicted by context.

The description herein of any aspect or embodiment of the invention using terms such as “comprising”, “having,” “including,” or “containing” with reference to an element or elements is intended to provide support for a similar aspect or embodiment of the invention that “consists of”, “consists essentially of”, or “substantially comprises” that particular element or elements, unless otherwise stated or clearly contradicted by context (e.g., a composition described herein as comprising a particular element should be understood as also describing a composition consisting of that element, unless otherwise stated or clearly contradicted by context).

1. A peptidic insulin receptor antagonist comprising an amino acid sequence that comprises a formula 1-type sequence having at least about 80% identity to Gly Ser Leu Asp Glu Ser Phe Tyr Asp Trp Phe Glu Arg Gln Leu Gly (SEQ ID NO:1) and a formula 6-type sequence according to the formula Leu Xaa Xaa Glu Trp Ala Xaa Xaa Gln Cys Glu Val Xaa Gly Arg Gly Cys Pro Ser,
wherein Xaa represents any amino acid residue; the formula 1-type sequence and formula 6-type sequence are positioned in an N-to-C terminus orientation in the amino acid sequence; and (a) the amino acid sequence comprises an amino acid sequence linker of at least one amino acid residue located between the formula 1-type and formula 6-type sequence, (b) the amino acid sequence is associated with a terminal derivatizing moiety that enhances stability of the insulin receptor antagonist in vivo, or (c) the amino acid sequence comprises an amino acid sequence linker of at least one amino acid residue located between the formula 1-type and formula 6-type sequence and the amino acid sequence is associated with a terminal derivatizing moiety that enhances stability of the insulin receptor antagonist in vivo.

2. The insulin receptor antagonist of claim 1, wherein the formula 6 sequence is not WLDEEWAQVQCEVYGRGCPS (SEQ ID NO: 14) or WLDQEWAWVQCEVYGRGCPS (SEQ ID NO:15).

3. The insulin receptor antagonist of claim 1, wherein the formula 1-type sequence is GSLDESFYDWFERQLG (SEQ ID NO:1).

4. The insulin receptor antagonist of claim 1, wherein the formula 6-type sequence is at least about 85% identical to SLEEEWAQIQCEVWGRGCPSY (SEQ ID NO:16).

5. The insulin receptor antagonist of claim 4, wherein the formula 6-type sequence is SLEEEWAQIQCEVWGRGCPSY (SEQ ID NO:16).

6. The insulin receptor antagonist of claim 1, wherein the formula 6-type sequence falls within the formula Leu Xaa₃ Xaa₄ Glu Trp Ala Xaa₈ Xaa₉ Gln Cys Glu Val Xaa₁₄ Gly Arg Gly Cys Pro Ser Xaa₂₁ (SEQ ID NO:17), wherein Xaa₃ is Asp or Glu; Xaa₄ is Cys, Asp, Glu, His, Lys, Asn, Gln, Arg, Ser, or Thr; Xaa₈ is any amino acid residue; Xaa₉ is Ile, Leu, Val, Met, Phe, Gly, or Ala; Xaa₁₄ is Trp, Phe, or Tyr; and Xaa₂₁ is either absent or selected from Ser, Thr, Asn, Gln, Tyr, Cys, or Gly.

7. The insulin receptor antagonist of claim 1, wherein the formula 6-type sequence falls within the formula Xaa₁Leu Xaa₃ Xaa₄ Glu Trp Ala Xaa₈ Xaa₉ Gln Cys Glu Val Xaa₁₄ Gly Arg Gly Cys Pro Ser Xaa₂₁ (SEQ ID NO:18), wherein Xaa₁ is Trp or Ser, Xaa₃ is Glu or Asp; Xaa₄ is Glu or Gln; Xaa₈ is Gln or Trp; Xaa₉ is Ile or Val; Xaa₁₄ is Trp or Tyr; and Xaa₂₁ is absent or Tyr (Formula 6b).

8. The insulin receptor antagonist of claim 1, wherein the formula 6-type sequence does not comprise the sequence SLEEEWAQIQCEVWGRGCPS (SEQ ID NO:19).

9. The insulin receptor antagonist of claim 1, wherein the amino acid sequence further comprises an amino acid linker of 1-7 amino acid residues positioned between the formula 1-type sequence and formula 6-type sequence.

10. The insulin receptor antagonist of claim 9, wherein the majority of the residues that form the linker are Gly or Ser residues.

11. The insulin receptor antagonist of claim 1, wherein the insulin receptor antagonist comprises a terminal derivatizing moiety.

12. The insulin receptor antagonist of claim 11, wherein the terminal derivatizing moiety comprises an amide-derivatized amino acid residue that forms the C-terminal end of, of or is located C-terminal to the C-terminal end of, the formula 6-type sequence.

13. An insulin receptor antagonist comprising an amino acid sequence according to the formula GSLDESFYDWFERQL-Z₁-SLEEEWAQIQCEVWGRGCPS-Z₂ (SEQ ID NO:20), wherein Z₁ represents an amino acid linker and Z₂ represents an optionally present terminal derivatizing moiety, an amino acid or amino acid sequence, a derivatized amino acid residue, or an amino acid sequence comprising a C-terminal derivatized amino acid residue.

14. An insulin receptor antagonist comprising compound S661 (GSLDESFYDWFERQLGGGSGGSSLEEEWAQIQCEVWGRGCPSY-amide).

15. A pharmaceutical composition comprising a therapeutically effective amount of an insulin receptor antagonist according to claim 1.

16. A composition according to claim 15, wherein the composition is formulated for subcutaneous injection, oral delivery, or pulmonal delivery.

17. A method of decreasing insulin receptor activity in mammalian cells comprising delivering a physiologically effective amount of an insulin receptor antagonist according to claim 1 to the cells so as decrease insulin receptor activity.

18. A method of treating hyperinsulinemia in a subject comprising delivering to the subject a therapeutically effective amount of a insulin receptor antagonist according to claim 1 to the subject so as to treat hyperinsulinemia.

19. A method of preventing or ameliorating hyperinsulinemia-related hypoglycemia in a subject comprising delivering to the subject a therapeutically effective amount of an insulin receptor antagonist according to claim 1.

20. (canceled)
